SEQUENCE LISTING (14 sequences)

SEQ ID NO: 1 - DNA, 30 bp, synthetic construct
  source 1..30: mol_type = other DNA
cttcagatgatgatcccaagccttctggcg

SEQ ID NO: 2 - DNA, 24 bp, synthetic construct
  source 1..24: mol_type = other DNA
aattggtgggcctgggaggacttg

SEQ ID NO: 3 - DNA, 22 bp, synthetic construct
  source 1..22: mol_type = other DNA
gttagagcgcgcgcttgataag

SEQ ID NO: 4 - DNA, 31 bp, synthetic construct
  source 1..31: mol_type = other DNA
aggtcattttgggggtttagctcagttggct

SEQ ID NO: 5 - DNA, 26 bp, synthetic construct
  source 1..26: mol_type = other DNA
aatggaggttaagggactcgaaccct

SEQ ID NO: 6 - DNA, 24 bp, synthetic construct
  source 1..24: mol_type = other DNA
cggctttgcaagccgagggtcaag

SEQ ID NO: 7 - DNA, 28 bp, synthetic construct
  source 1..28: mol_type = other DNA
tcaygaagacctcacagtaaaaataggt

SEQ ID NO: 8 - DNA, 26 bp, synthetic construct
  source 1..26: mol_type = other DNA
gatccagacaactgttcaaactgatg

SEQ ID NO: 9 - DNA, 25 bp, synthetic construct
  source 1..25: mol_type = other DNA
gtctagccacagtgaaatctcgatg

SEQ ID NO: 10 - DNA, 24 bp, synthetic construct
  source 1..24: mol_type = other DNA
cggctttgcaagccgagggtcaag

SEQ ID NO: 11 - DNA, 26 bp, synthetic construct
  source 1..26: mol_type = other DNA
taaaytggtgggcctgggaggacttg

SEQ ID NO: 12 - DNA, 26 bp, synthetic construct
  source 1..26: mol_type = other DNA
ttccttccctgaagggattcactcag

SEQ ID NO: 13 - DNA, 27 bp, synthetic construct
  source 1..27: mol_type = other DNA
ttaagcccagcagcctatctgacaccc

SEQ ID NO: 14 - DNA, 32 bp, synthetic construct
  source 1..32: mol_type = other DNA
gaaaagcctgtttaccaaggagcttgaacatg

(19) United States
(12) Patent Application Publication
(10) Pub. No.: US 2023/0123800 A1
(43) Pub. Date: Apr. 20, 2023

(54) DIGITAL PCR ASSAYS FOR DETECTION OF PATHOGENS

(71) Applicant: GALAXY DIAGNOSTICS, INC., RESEARCH TRIANGLE PARK, NC (US)

(72) Inventors: RICARDO G MAGGI, Apex, NC (US); JENNIFER C. MILLER, CARY, NC (US)

(21) Appl. No.: 18/046,342

(22) Filed: Oct. 13, 2022

Related U.S. Application Data
(63) Continuation-in-part of application No. PCT/US2021/027427, filed on Apr. 15, 2021.
(60) Provisional application No. 63/010,281, filed on Apr. 15, 2020.

(51) Int. Cl. C12Q 1/689; C12Q 1/686

(52) U.S. Cl. CPC C12Q 1/689; C12Q 1/686

(57) ABSTRACT

Provided herein are compositions, methods, and kits for detecting the presence of and/or identifying fastidious microorganisms, such as Bartonella spp., Borrelia spp., Anaplasma spp., Ehrlichia spp., Babesia/Theileria spp., Rickettsia spp., and/or Mycoplasma spp., in a sample. The compositions, methods and kits of the inventive concept include use of droplet digital PCR (ddPCR) assays for the detection and quantitation of the fastidious microorganisms.

### CROSS-REFERENCE TO RELATED APPLICATIONS

This application claims the benefit of U.S. Provisional Application Ser. No. 63/010,281, filed Apr. 15, 2020, which is incorporated herein by reference in its entirety for its disclosures.

### STATEMENT OF GOVERNMENT SUPPORT

This invention was made with government support under Grant No. R43HL144161 awarded by the National Institutes of Health/National Heart, Lung, and Blood Institute (NIH/NHLBI) The government has certain rights in the invention.

### STATEMENT REGARDING ELECTRONIC FILING OF A SEQUENCE LISTING

A Sequence Listing in ASCII text format, submitted under 37 C.F.R. § 1.821, entitled 191202-00006 ST25 txt, 3,696 bytes in size, generated on Apr. 15, 2021 and filed via EFS-Web, is provided in lieu of a paper copy. The Sequence Listing is incorporated herein by reference into the specification for its disclosures.

### FIELD

The present inventive concept is related to improved methods, compositions, and kits for detecting and/or identifying the presence of a fastidious microorganism(s) in a sample, and methods, compositions and kits for detecting and/or identifying the presence of a fastidious microorganism(s) in a subject.

### BACKGROUND

In recent years, the development of droplet digital PCR (ddPCR), a new molecular technology, has enabled an unprecedented expansion of DNA amplification capabilities for research and diagnostic applications, not just in the area of cancer and gene expression, but also in the areas of infectious disease diagnosis. ddPCR is a powerful molecular technique that uses a water-oil emulsion technology driven by microfluidics and surfactant chemistry to massively partition samples into 15,000 to 20,000 1 ηL sized droplets prior to performing DNA amplification. DNA targets within the original sample are randomly localized within these droplets, after which amplification of DNA within each drop is recorded using fluorescently labeled probe detection. The number and fluorescent output of each droplet is read in a manner similar to flow cytometry, where each individual droplet is then identified as being positive or negative for the template (pathogen gene target) of interest. Two significant advantages of this technology, in addition to its increased efficiency, are its unparalleled precision and simplified absolute quantification that does not require the use of a standard curve. In addition, by sequestering target DNA among individual droplets, ddPCR reduces the impact of inhibitory substances, including hemoglobin, heparin, and high concentrations of host DNA, that typically compete during conventional and real-time qPCR (Table 1).

The development of commercial ddPCR systems with integrated technologies, including automated sample partitioning, digital counting of nucleic acid targets, and increased droplet count, has significantly streamlined the DNA detection process, making it more efficient and standardized for clinical diagnostic testing.

Bartonella species are slow growing, fastidious, facultative Gram-negative intracellular bacteria that infect a variety of mammalian hosts including production and companion animals, wildlife, and humans, via arthropod vectors, animal bites, blood transfusion, or organ transplantation. Bartonellosis is associated with a wide variety of human pathologies including endocarditis, cat scratch disease (CSD), bacillary angiomatosis (BA), bacillary peliosis (BP), and neurological diseases. Current detection methods for the diagnosis of bartonellosis include serological immunofluorescence assays (IFA), polymerase chain reaction (PCR), and blood cultures, all of which have sensitivity limitations. Despite the wide availability of these assays, laboratory diagnosis of bartonellosis remains extremely difficult. This is due to the fastidious nature of the bacteria (exhibit a doubling time between 21 and 24 hrs), their complex growth requirements (i.e. requirement of growth factors such as hemoglobin or heme groups), and the relapsing, low-level bacteremia associated with Bartonella spp. infection (resulting in very low to undetectable levels of bacteria within blood, tissues, and body fluids). In addition, Bartonella spp. can invade several cell types, evade the host's immune system (often leading to long delays in seroconversion and negative serology test results) and subvert cellular functions leading to vasoproliferative or chronic (granulomatous) inflammatory disorders.

Borreliosis, accounting for almost 75% of vector-borne diseases (VBD), is the most common VBD in the USA, affecting over 300,000 people a year. The disease is caused by spirochetal bacteria within the genus Borrelia, of which B. burgdorferi is the most recognized pathogenic species in the northern hemisphere. Diagnosis of acute borreliosis is based upon symptoms (e.g. headache, fatigue, malaise, muscle pain), clinical signs (presence of an erythema migrans (EM) skin rash), and two-tier serological diagnostic testing (a positive or equivocal first tier IgM or IgG enzyme immunoassay (EIA) or immunofluorescent assay (IFA) result is confirmed by a positive second tier or reflex Western blot test). People treated with appropriate antibiotics (i.e., doxycycline, amoxicillin, or cefuroxime axetil) in the early stages of borreliosis usually recover rapidly and completely. However, if early therapeutic intervention is not obtained (chronic borreliosis) or if treatment fails (Post-treatment Lyme disease syndrome (PTLDS)) the infection can spread to the nervous system, joints, and heart. Chronic borreliosis and PTLDS are commonly associated with persistent symptoms and signs such as musculoskeletal problems, fatigue, cardiac presentations, cognitive dysfunction, headaches, sleep disturbance, and other neurologic features such as demyelinating disease, peripheral neuropathy, neurodegenerative disease, and neuropsychiatric presentations. In addition, concurrent infection with other vector-borne pathogens, including bacteria belonging to the genus Bartonella, further complicates and confounds the clinical diagnosis and treatment of diseases caused by this diverse group of pathogens. In recent years, bartonellosis has been recognized as an emerging/re-emerging zoonotic infectious disease caused by numerous mammalian reservoir-adapted Bartonella species, with at least 18 Bartonella spp. implicated as causative agents of disease in animals or humans.

The current diagnostic gold standard for documentation of Bartonella spp. infection (the Bartonella species ePCR® platform) includes Bartonella DNA amplification from blood (or alternatively, cerebrospinal fluid, joint fluid and pathological effusions) before and after sample enrichment in a specialized formulated liquid broth, such as BAPGM (Bartonella Alpha Proteobacteria Growth Medium). Unfortunately, the sensitivity associated with conventional and qPCR methodologies utilized for Bartonella spp. DNA detection are poor due to the low bacterial load present within most biological samples, the long time required for bacterial growth in enrichment culture, and the presence of PCR-inhibitory components (such as anti-coagulants, hemoglobin, and high concentrations of host DNA) found within blood and tissues. As such, there is still a need for improved methods and tools for detection and documentation of fastidious microorganisms, such as Bartonella and Borrelia, among others.

### SUMMARY

According to an aspect of the inventive concept, provided is a method of detecting a fastidious microorganism in a sample including: performing a nucleic acid amplification on the sample for a nucleic acid sequence that can identify the presence of the fastidious microorganism, wherein performing the nucleic acid amplification includes distributing nucleic acids from the sample into a plurality of partitions, and performing nucleic acid amplification on the plurality of partitions; and detecting whether a nucleic acid amplification product is produced in the nucleic acid amplification, wherein presence of the nucleic acid amplification product is indicative of the fastidious microorganism being present in the sample.

According to another aspect of the inventive concept, provided is a method of identifying a fastidious microorganism in a sample including: performing a nucleic acid amplification for nucleic acid sequences associated with a fastidious microorganism species or genus on a sample obtained from a subject suspected of being infected by a fastidious microorganism, wherein performing the nucleic acid amplification includes distributing nucleic acids from the sample into a plurality of partitions, and performing nucleic acid amplification on the plurality of partitions, wherein the plurality of partitions is generated using a microfluidic process and/or a droplet generating process; and analyzing nucleic acid amplification products derived from the nucleic acid amplification,

According to another aspect of the inventive concept, provided is a method of detecting a fastidious microorganism in a sample derived from a subject including: performing a nucleic acid amplification for a nucleic acid sequence that can identify the presence of the fastidious microorganism on the nucleic acids extracted from the sample; and detecting whether a nucleic acid amplification product is produced in the nucleic acid amplification, wherein performing the nucleic acid amplification and detecting whether a nucleic acid amplification product is produced includes analysis by a water-oil emulsion droplet polymerase chain reaction system, such as droplet digital PCR (ddPCR), and wherein presence of the nucleic acid amplification product is indicative of the fastidious microorganism being present in the subject.

According to another aspect of the inventive concept, provided is a method of identifying a fastidious microorganism in a sample derived from a subject including: performing a nucleic acid amplification for nucleic acid sequences associated with a fastidious microorganism species on the nucleic acids extracted from the sample; and detecting whether a nucleic acid amplification product is produced in the nucleic acid amplification, wherein performing the nucleic acid amplification and detecting whether a nucleic acid amplification product is produced includes analysis by droplet digital PCR (ddPCR), and wherein analysis by ddPCR of the nucleic acid amplification products provides information regarding identity of the fastidious microorganism infecting the subject.

### BRIEF DESCRIPTION OF THE DRAWINGS

FIG. 1: ddPCR detection and droplet distribution of four Bartonella DNA species spiked into naïve human blood DNA. Note. Channel 1: Bartonella DNA amplification; Channel 2: Human DNA (housekeeping) amplification.

FIG. 2: Inclusion of HindIII within the ddPCR pre-mix results in optimal droplet resolution, distribution, and separation of Bartonella and housekeeping gene populations. (A) ddPCR amplification signal (amplitude) resolution when no restriction enzyme is used to digest the DNA sample template, (B) when restriction enzyme (Hindu) is used to digest DNA within the sample (pre-digestion) before inclusion as template, (C) when the restriction enzyme is added during pre-mix preparation. Note. Channel 1: Bartonella DNA amplification; Channel 2: Human DNA (housekeeping) amplification.

FIG. 3: ddPCR detection and droplet distribution of DNA from 14 Bartonella species/strains spiked into naïve human blood. Abv: BhSA2: B. henselae San Antonio 2; BhHI: B. henselae Houston I; Bvb TI: B. v. berkhoffii genotype I; Bvb TII: B. v. berkhoffii genotype II; Bvb TIII: B. v. berkhoffii genotype III; Bq: B. quintana Fuller; Bk: B. koehlerae; Bc: B. clarredgeiae; Bmelo: B. melophagi; Beliz: B. elizabethae. Note. Channel 1: Bartonella DNA amplification; Channel 2: Human DNA (housekeeping) amplification.

FIGS. 4A and 4B: ddPCR detection and droplet distribution of dual Bartonella species DNAs spiked at equal concentrations into naïve human blood. FIG. 4A: B. henselae San Antonio 2 (BhSA2) and B. v. berkhoffii genotype II (Bvb TII); FIG. 4B: B. quintana Fuller (Bq) and B. v. berkhoffii genotype II (Bvb TII). Note. Channel 1: Bartonella DNA amplification; Channel 2: Human DNA (housekeeping) amplification.

FIG. 5: ddPCR detection and droplet distribution of DNA obtained from human epithelial mammary cells (MCF10A) intracellularly infected in vitro with B. henselae SA2. The images show positive and negative dot distribution for samples collected at 24, 48, and 72 hrs post infection. Multiplicities of infection (MOIs) of 1:1 and 10:1 are compared with naïve uninfected cells (results on MOIs of 30:1 and 100:1 not shown). Note. Channel 1: Bartonella DNA amplification; Channel 2: Human DNA (housekeeping) amplification.

FIG. 6: Droplet distribution of intracellular Borrelia (circles in upper right-hand quadrant) and Braf housekeeping (circles in lower right-hand quadrant) gene DNA within DH82 cells infected with B. burgdorferi B31 at an MOI of 10:1. Samples were collected at 24, 48, and 72 hrs post-infection.

FIG. 7: Droplet distribution detection amplitudes for Borrelia DNA (Channel 1) and the host housekeeping Braf gene (Channel 2) in spiked blood samples. Borrelia DNA was spiked at concentrations ranging from 1 to 0.001 pg/ul in naïve dog blood.

FIG. 8: Droplet distribution of Bartonella (dots in upper left-hand quadrant) and Borrelia (dots in lower right-hand quadrant) species DNA spiked into naïve human blood. Single spikes were performed using B. henselae, B. quintana, B. v. berkhoffi genotype II (BvbTII), or B. burgdorferi B31. Dual species spikes were performed using B. henselae+B. burgdorferi, B. quintana+B. burgdorferi (not shown), or B. v. berkhoffi genotype II+B. burgdorferi. No amplification (blue or green dots) was observed in either channel when DNA from the same naïve human blood sample was assayed.

### DETAILED DESCRIPTION

The foregoing and other aspects of the present inventive concept will now be described in more detail with respect to other embodiments described herein. It should be appreciated that the inventive concept can be embodied in different forms and should not be construed as limited to the embodiments set forth herein. Rather, these embodiments are provided so that this disclosure will be thorough and complete, and will fully convey the scope of the inventive concept to those skilled in the art.

The terminology used in the description of the inventive concept herein is for the purpose of describing particular embodiments only and is not intended to be limiting of the inventive concept. As used in the description of the inventive concept and the appended claims, the singular forms “a”, “an” and “the” are intended to include the plural forms as well, unless the context clearly indicates otherwise. Additionally, as used herein, the term “and/or” includes any and all combinations of one or more of the associated listed items and may be abbreviated as “/”.

The term “comprise,” as used herein, in addition to its regular meaning, may also include, and, in some embodiments, may specifically refer to the expressions “consist essentially of” and/or “consist of.” Thus, the expression “comprise” can also refer to, in some embodiments, the specifically listed elements of that which is claimed and does not include further elements, as well as embodiments in which the specifically listed elements of that which is claimed may and/or does encompass further elements, or embodiments in which the specifically listed elements of that which is claimed may encompass further elements that do not materially affect the basic and novel characteristic(s) of that which is claimed. For example, that which is claimed, such as a composition, formulation, method, system, etc. “comprising” listed elements also encompasses, for example, a composition, formulation, method, kit, etc. “consisting of,” i.e., wherein that which is claimed does not include further elements, and a composition, formulation, method, kit, etc. “consisting essentially of,” i.e., wherein that which is claimed may include further elements that do not materially affect the basic and novel characteristic(s) of that which is claimed.

The term “about” generally refers to a range of numeric values that one of skill in the art would consider equivalent to the recited numeric value or having the same function or result. For example, “about” may refer to a range that is within ±1%, ±2%, ±5%, ±10%, ±15%, or even±20% of the indicated value, depending upon the numeric values that one of skill in the art would consider equivalent to the recited numeric value or having the same function or result. For example, about 10 may include a range, for example, from 8.5 to 11.5. Furthermore, in some embodiments, a numeric value modified by the term “about” may also include a numeric value that is “exactly” the recited numeric value. In addition, any numeric value presented without modification will be appreciated to include numeric values “about” the recited numeric value, as well as include “exactly” the recited numeric value. Similarly, the term “substantially” means largely, but not wholly, the same form, manner or degree and the particular element will have a range of configurations as a person of ordinary skill in the art would consider as having the same function or result. When a particular element is expressed as an approximation by use of the term “substantially,” it will be understood that the particular element forms another embodiment.

Unless otherwise defined, all technical and scientific terms used herein have the same meaning as commonly understood by one of ordinary skill in the art to which this inventive concept belongs.

All publications, patents, and patent applications mentioned in this specification are herein incorporated by reference to the same extent as if each individual publication, patent, or patent application was specifically and individually indicated to be incorporated by reference.

In general, described herein are methods, compositions, and kits for detecting and/or identifying the presence of a fastidious microorganism(s) in a sample, such as a sample obtained from a subject. For example, in some embodiments, the methods, compositions, and kits of the present inventive concept may be used to identify the presence and/or nature of an infection, disease, and/or disorder in a sample and/or subject resulting from the presence of a particular fastidious microorganism. In some embodiments, the methods, compositions, and kits of the present inventive concept may be used to identify and/or characterize the particular fastidious microorganism or microorganisms that may be present in a sample and/or subject.

As used herein, a “fastidious” organism (including a fastidious microorganism) is an organism that is recalcitrant to culturing using conventional media (e.g., sheep's blood agar, chocolate agar, bile salt agar, Dulbecco's modified Eagle's medium, Eagle's medium, F12 medium, and the like), and will typically have more stringent requirements with respect to the chemical composition of the media. For example, the organism may require that particular organic molecules be provided in the medium due to defective or insufficient enzymatic machinery for synthesis of nutrients such as vitamins, amino acids, lipids, cofactors such as NAD, NADH, nucleosides or free radical scavengers. Alternatively, or additionally, the fastidious organism may have a requirement that particular organic molecules be supplied in the medium or, conversely, have an intolerance for certain components (e.g., sugars) used in many media.

The term “microorganism” as used herein has its conventional meaning in the art and includes bacteria, protozoa, yeasts, molds, and viruses. In some embodiments, the microorganism is a bacterium, for example, a Proteobacterium (including subgroups, alpha, beta, delta and gamma), such as, for example, an Alphaproteobacteria (e.g., Bartonella spp., Brucella spp., Ehrlichia spp., Rasbo bacteria, Afipia, Anaplasma, and the like), including nanobacteria. In other embodiments, the microorganism may be a Corynebacterium, a bacterium belonging to the group Apicomplexa, for example, Babesia/Theileria, Firmicutes, Fusobacteria, Planctomycetales, Spirochete, for example, Borrelia, Burkholderia pickettii, Streptococcus thermophillis, or division Archaea bacteria. In some embodiments, the microorganism belongs to the group Mollicutes, for example, Mycoplasma spp., and the like. In some embodiments, the microorganism may be, for example, Bartonella spp., Borrelia spp., Anaplasma spp., Ehrlichia spp., Babesia/Theileria spp., Rickettsia spp., or Mycoplasma spp. In some embodiments, the microorganism may be Bartonella spp., for example, B. henselae, B. quintana, B. alsatica, B. elizahethae, B. koehlerae, B. vinsonii subsp. berkhoffii genotype I, B. vinsonii subsp. berkhoffii genotype II, B. vinsonii subsp. berkhoffii genotype III, B. vinsonii subsp. berkhoffii genotype IV, B. vinsonii subsp. vinsonii, and/or B. clarridgeiae. In some embodiments, the microorganism may be Borrelia spp., for example, B. burgdorferi, B. bissettii, B. garinii, B. mayonii, B. hermsii, B. parkeri, B. turicatae, B. miyamotoi, and/or B. afzelii.

Microorganisms according to the present inventive concept are typically mammalian and/or avian pathogens, in particular embodiments, mammalian pathogens. Alternatively, the microorganism may be a reptilian, piscine, amphibian, insect and/or plant pathogen. Exemplary microorganisms are insect-borne or arachnid-borne pathogens. The term “pathogen”, as used herein, refers to microorganisms that are of clinical relevance because they are believed to be associated with, correlated with, and/or indicative of a disease state or disorder. As used herein, a pathogen, such as, but not limited to, Bartonella spp. or Borrelia spp., and/or infections resulting from a pathogen, such as, but not limited to, bartonellosis or borreliosis, are “associated” with a disease state or disorder if it is believed to be a contributing or underlying factor in the development of a disease state or disorder, or it may be more prevalent in individuals with a particular disease or disorder (e.g., Bartonellosis in patients infected with the Human Immunodeficiency Virus).

In some embodiments, the pathogen, and/or infections resulting from the pathogen, may be associated with, for example, chronic fatigue syndrome, cancer, hypertension, heart disease (e.g., endocarditis), feline urologic disease, hyperadrenalcorticism, mastitis, polyarthritis, immune-mediated hemolytic anemia, thrombocytopenia, cystic fibrosis, cat scratch disease, renal disease, liver disease, prostate disease, or central nervous system disorders.

Alternatively, the microorganism is found in a sample taken from a mammalian or avian source, but is not necessarily a pathogen. The terms referring to a sample from a “mammalian subject,” “mammalian source,” or “a mammalian sample” indicate that the sample has been derived from a mammal or a mammalian tissue, organ, cell culture, body fluid or waste product. Likewise, the terms referring to a sample from an “avian subject,” “avian source,” or “an avian sample” indicate that the sample has been derived from an avian or an avian tissue, organ, cell culture, body fluid or waste product. In some embodiments, the sample is a body fluid sample (including tissue fluids), a tissue sample, or an organ sample. Exemplary body fluid samples according to the inventive concept include but are not limited to blood, plasma, serum, milk, urine, cerebrospinal fluid (CSF), pleural fluid, pulmonary mucus, sputum, transudates, modified transudates, exudates, chest fluid, abdominal fluid, synovial fluid, peritoneal fluid, lymph, and effusions. Tissue or organ samples (e.g., biopsies or swabs) may be from any tissue or organ in the body and include but are not limited to: skin, liver, heart, kidney, brain and other tissues of the central nervous system, ear, nasal tissue, airway passages, lungs, prostate, ovary, testis, uterus, pancreas, spleen, stomach, esophagus, mouth, intestines, colon, rectum, eye, ear, vagina, cervix, urinary tract, and muscle. In other embodiments, the sample is from a waste product such as a fecal or mucus sample. In still other embodiments, the sample is from an animal product such as meat, eggs, milk or feathers.

The sample may also be derived from an insect or arachnid that is a vector for the microorganism.

Mammals according to and that may benefit from the present inventive concept include but are not limited to canine, felines, bovines, caprines, equines, ovines, porcines, rodents, lagomorphs, primates, and the like, and encompass mammals in utero. In some embodiments mammals according to and that may benefit from the present inventive concept include, but are not limited to, felines, canines, rodents, bovines, equines and humans. In some embodiments, the mammal or mammalian subject may be a feline, a canine, a rodent, or a human.

Illustrative avians according to and that may benefit from the present inventive concept include but are not limited to chickens, ducks, turkeys, geese, quail, pheasant, ratites (e.g., ostrich) and domesticated birds (e.g., parrots and canaries), and include birds in ovo. In some embodiments, the avian or avian subject according to and that may benefit from the present inventive concept include chickens and turkeys.

Other hosts include reptilians (e.g., crocodiles, snakes, turtles), pisces (e.g., fish in commercial hatcheries) and amphibians (e.g., frogs).

### Amplification

The methods, compositions, and kits for detecting and/or identifying the presence of a fastidious microorganism(s) in a sample, such as a sample obtained from a subject, of the present inventive concept may include performing a nucleic acid amplification on the sample, or on nucleic acids extracted from the sample. Nucleic acids can be extracted from a sample by means available to one of ordinary skill in the art. Nucleic acids, as described herein, include polynucleotides, e.g., DNA, RNA, mitochondrial DNA, genomic DNA, mRNA, siRNA, miRNA, cRNA, single-stranded DNA, double-stranded DNA, single-stranded RNA, double-stranded RNA, tRNA, rRNA, cDNA, etc., or a hybrid, where the nucleic acid can contain any combination of deoxyribo- and ribo-nucleotides, and any combination of bases, including uracil, adenine, thymine, cytosine, guanine, inosine, xathanine hypoxathanine, isocytosine, isoguanine, etc.

The sample, and/or nucleic acids extracted from the sample may be processed to render it competent for amplification. Exemplary sample processing can include lysing cells of the sample to release nucleic acid, purifying the sample (e.g., to isolate nucleic acid from other sample components, which may inhibit amplification), diluting/concentrating the sample, and/or combining the sample with reagents for amplification, such as a DNA/RNA polymerase (e.g., a heat-stable DNA polymerase for PCR amplification), dNTPs (e.g., dATP, dCTP, dGTP, and dTTP (and/or dUTP)), a primer set for each allele sequence or polymorphic locus to be amplified, probes (e.g., fluorescent probes, such as TAQMAN probes or molecular beacon probes, among others) capable of hybridizing specifically to each allele sequence to be amplified, Mg²⁺, DMSO, BSA, a buffer, or any combination thereof, among others. In some examples, the sample may be combined with a restriction enzyme, uracil-DNA glycosylase (UNG), reverse transcriptase, or any other enzyme of nucleic acid processing.

In some embodiments, the nucleic acid amplification may include polymerase chain reaction (PCR), reverse-transcription PCR, quantitative PCR, real-time PCR, isothermal amplification, linear amplification, or isothermal linear amplification, quantitative fluorescent PCR (QF-PCR), multiplex fluorescent PCR (MF-PCR), single cell PCR, restriction fragment length polymorphism PCR(PCR-RFLP), PCR-RFLP/RT-PCR-RFLP, hot start PCR, nested PCR, in situ colony PCR, in situ rolling circle amplification (RCA), bridge PCR (bPCR), picotiter PCR, digital PCR, droplet digital PCR, or emulsion PCR (emPCR). Other suitable amplification methods include ligase chain reaction (LCR (oligonucleotide ligase amplification (OLA)), transcription amplification, cycling probe technology (CPT), molecular inversion probe (MIP)PCR, self-sustained sequence replication, selective amplification of target polynucleotide sequences, consensus sequence primed polymerase chain reaction (CP-PCR), arbitrarily primed polymerase chain reaction (AP-PCR), transcription mediated amplification (TMA), degenerate oligonucleotide-primed PCR (DOP-PCR), multiple-displacement amplification (MDA), strand displacement amplification (SDA), and nucleic acid based sequence amplification (NABSA). Other amplification methods that can be used herein include those described in U.S. Pat. Nos. 5,242,794; 5,494,810; 4,988,617; and 6,582,938, each of which is hereby incorporated herein by reference in its entirety.

In some embodiments, nucleic acid amplification includes digital PCR. It will be appreciated that digital PCR may include any method, process, and/or protocol, using instruments and/or kits associated with performing such, that can discretely amplify and quantitate a nucleic acid(s) within individual partitions of a sample. In some embodiments, the individual partitions for a digital PCR may be generated by a microfluidic process, such as by using a microfluidic device, and/or by a droplet generating process. Generation of individual partitions by a microfluidic process, such as by using a microfluidic device, and/or a droplet generating process to provide a plurality of partitions in the form of droplets and performing nucleic acid amplification thereon has been more particularly described in the art as “droplet digital PCR.” The droplets generated for droplet digital PCR may be provided in, for example, a water-in-oil emulsion. In some embodiments, the methods, processes, and/or protocols, and instruments and/or kits for performing nucleic acid amplification on partitions in the form of droplets generated using a microfluidic device/process and/or a droplet generating process, are commercially available, for example, but not limited to, those provided by Bio-Rad, 10× Genomics, Qiagen, and/or ThermoFisher. In an exemplary embodiment, nucleic acid amplification includes droplet digital PCR (ddPCR™) using Bio-Rad's QX100™ or QX200™ Droplet Digital PCR systems, and analysis of nucleic acid amplification products produced by the same, but is not limited thereto.

Nucleic acid amplification in the methods, compositions, and kits for detecting and/or identifying the presence of a fastidious microorganism(s) in a sample, such as a sample obtained from a subject, of the present inventive concept may include separating a sample including nucleic acids/polynucleotides, or nucleic acids extracted from a sample, into a plurality of partitions, e.g., droplets. Reagents necessary for nucleic acid amplification, e.g., oligonucleotide primers, oligonucleotide probes, NTPs/dNTPs, polymerases, etc., may be provided to each of the plurality of partitions, and nucleic acid amplification may be performed on each of the plurality of partitions. In some embodiments, reagents for nucleic acid amplification may be bundled within a partition, e.g., an aqueous phase of an emulsion, e.g., a droplet. Partitions containing nucleic acid amplification reagents/components may be merged/fused with partitions containing a sample/nucleic acid extracted from a sample, and nucleic acid amplification performed on the merged/fused partitions.

A microfluidic device can be used to merge nucleic acid amplification reagent-containing partitions with sample/nucleic acid extract-containing partitions such that every sample/nucleic acid-containing partition includes nucleic acid amplification components/reagents. The number of partitions provided is not particularly limited. In some embodiments, the number of different sample/polynucleotide-containing partitions, e.g., in a sequencing reaction, can be about, more than about, less than about, or at least about 1000, 5000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, or 10,000,000 partitions. In some embodiments, about 1000 to about 10,000, about 10,000 to about 100,000, about 10,000 to about 500,000, about 100,000 to about 500,000, about 100,000 to about 1,000,000, about 500,000 to about 1,000,000, about 1,000,000 to about 5,000,000, or about 1,000,000 to about 10,000,000 partitions may be generated in the methods as set forth herein.

Methods for generating droplets are described, for example, in U.S. Patent Application Publication No. 2011/0053798, hereby incorporated herein by reference in its entirety. In some embodiments, for example, an inner droplet (or partition) can be fused with an outer droplet (or partition) by heating/cooling to change temperature, applying pressure, altering composition (e.g., via a chemical additive), applying acoustic energy (e.g., via sonication), exposure to light (e.g., to stimulate a photochemical reaction), applying an electric field, or any combination thereof. In some cases, the inner droplet may fuse to the outer droplet spontaneously. The treatment may be continuous or may vary temporally (e.g., pulsatile, shock, and/or repetitive treatment). The treatment may provide a gradual or rapid change in an emulsion parameter, to effect steady state or transient initiation of droplet fusion. The stability of the partitions, and their responsiveness to a treatment to induce droplet fusion, may be determined during their formation by selection of an appropriate surfactant type, surfactant concentration, critical micelle concentration, ionic strength, etc., for one or more phases of the inner/outer partition.

Fusion may occur spontaneously, such that no treatment, other than a sufficient time delay (or no delay), is necessary before processing fused droplets. Alternatively, the inner/outer droplet may be treated to controllably induce fusion of droplets to form assay mixtures.

The emulsion resulting from the fusion may be processed. Processing may include subjecting the emulsion to any condition or set of conditions under which at least one reaction of interest can occur (and/or is stopped), and for any suitable time period. Accordingly, processing may include maintaining the temperature of the emulsion near a predefined set point, varying the temperature of the emulsion between two or more predefined set points (such as thermally cycling the emulsion), exposing the emulsion to light, changing a pressure exerted on the emulsion, adding at least one chemical substance to the emulsion, applying an electric field to the emulsion, or any combination thereof, among others.

Signals may be detected from the emulsion after and/or during processing. The signals may be detected optically, electrically, chemically, or a combination thereof, among others. The detected signals may include test signals that correspond to at least one reaction of interest performed in the emulsion. Alternatively, or in addition, the detected signals may include code signals that correspond to codes present in the emulsion. Test signals and code signals generally are distinguishable and may be detected using the same or distinct detectors. For example, the test signals and code signals each may be detected as fluorescence signals, which may be distinguishable based on excitation wavelength (or spectrum), emission wavelength (or spectrum), and/or distinct positions in a fused droplet (e.g., code signals may be detectable as more localized than test signals with respect to fused droplets), among others. As another example, the test signals and code signals may be detected as distinct optical characteristics, such as test signals detected as fluorescence and code signals detected as optical reflectance. As a further example, the test signals may be detected optically and the code signals electrically, or vice versa.

Partitions can be formed by any mode of separating that can be used for digital PCR. A partition can be a microfluidic channel, a well on a nano- or microfluidic device or on a microtiter plate, or a reaction chamber in a microfluidic device. A partition can be an area on an array surface. A partition can be an aqueous phase of an emulsion (e.g., a droplet).

In some embodiments, the present inventive concept includes compositions, methods, and kits for manipulation of genetic material in droplets, e.g., using droplet digital PCR. The droplets described herein can include emulsion compositions (or mixtures of two or more immiscible fluids) described in U.S. Pat. No. 7,622,280, and droplets generated by devices described in International Application Publication No. WO/2010/036352, each of which is hereby incorporated herein by reference in its entirety. The term emulsion, as used herein, can refer to a mixture of immiscible liquids (such as oil and water). Oil-phase and/or water-in-oil emulsions can allow for the compartmentalization of reaction mixtures within aqueous droplets. In some embodiments, the emulsions can include aqueous droplets within a continuous oil phase. In other embodiments, the emulsions provided herein are oil-in-water emulsions, wherein the droplets are oil droplets within a continuous aqueous phase. The droplets provided herein can be used to prevent mixing between compartments, and each compartment can protect its contents from evaporation and coalescing with the contents of other compartments. One or more enzymatic reactions can occur in a droplet.

Methods of generating partitions/droplets, additives, preparation of primers, probes, polymerases for PCR, etc. are described, for example, in U.S. Pat. No. 9,347,059, hereby incorporated herein by reference in its entirety.

Splitting a sample into small reaction volumes as described herein can enable the use of reduced amounts of reagents, thereby lowering the material cost of the analysis. Reducing sample complexity by partitioning can also improve the dynamic range of detection, since higher-abundance molecules can be separated from low-abundance molecules in different compartments, thereby allowing lower-abundance molecules greater proportional access to reaction reagents, which in turn can enhance the detection of lower-abundance molecules.

A computer can be used to store and process the data. A computer-executable logic can be employed to perform such functions as grouping and/or analyzing the data. A computer can be useful for displaying, storing, retrieving, or calculating diagnostic results from the molecular profiling; displaying, storing, retrieving, or calculating raw data; or displaying, storing, retrieving, or calculating any sample or patient information useful in the methods described herein. Provided herein are systems including computer readable instructions for performing methods described herein. Provided herein are computer readable medium including instructions which, when executed by a computer, cause the computer to perform methods described herein.

Also provided herein are kits for performing methods of the present inventive concept as described herein. The kits can include one or more restriction enzymes, endonucleases, exonucleases, ligases, polymerases, RNA polymerases, DNA polymerases, reverse transcriptases, topoisomerases, kinases, phosphatases, buffers, salts, metal ions, reducing agents, BSA, spermine, spermidine, glycerol, oligonucleotides, primers, probes, or labels (e.g., fluorescent labels). The kits can include one or more sets of instructions.

In some embodiments, a reference sequence that is present at two copies per diploid genome can be used, e.g., a housekeeping gene (e.g., a gene that is required for the maintenance of basic cellular function). Dividing the concentration or amount of the target by the concentration or amount of the reference can yield an estimate of the number of target copies per genome.

A housekeeping gene that can be used as reference in the methods described herein can include a gene that encodes a transcription factor, a transcription repressor, an RNA splicing gene, a translation factor, tRNA synthetase, RNA binding protein, ribosomal protein, RNA polymerase, protein processing protein, heat shock protein, histone, cell cycle regulator, apoptosis regulator, oncogene, DNA repair/replication gene, carbohydrate metabolism regulator, citric acid cycle regulator, lipid metabolism regulator, amino acid metabolism regulator, nucleotide synthesis regulator, NADH dehydrogenase, cytochrome C oxidase, ATPase, mitochondrial protein, lysosomal protein, proteosomal protein, ribonuclease, oxidase/reductase, cytoskeletal protein, cell adhesion protein, channel or transporter, receptor, kinase, growth factor, tissue necrosis factor, etc. Specific examples of housekeeping genes that can be used in the methods described include, e.g., human hydroxymethylbilane synthase (HMBS), or BRAF genes.

### Detection

As a further aspect, the present inventive concept provides methods for detecting a fastidious microorganism, as described herein, in a sample and/or subject, for example, a sample derived from a subject that may be suspected of having an infection from a particular fastidious microorganism. The fastidious microorganism may be detected by detection of microbial nucleic acids, e.g., by direct sequencing, nucleic acid amplification, hybridization to probes, and the like, and analysis of the nucleic acids/nucleic acid amplification products detected. In some embodiments, detecting the presence of the fastidious microorganism may include nucleic acid amplification, for example, as described hereinabove, and detection of nucleic amplification products.

Methods of detecting nucleic acids are well-known in the art and may include specific hybridization of the probe to sequences characteristic for the fastidious microorganism and, for example, detecting fluorescence emission from a fluorescently tagged or labeled oligonucleotide probe that hybridizes to a nucleic acid and/or a nucleic acid amplification product and gives off/releases fluorescence emission as a result of hybridization with the nucleic acid and/or the nucleic acid amplification product.

Any measurement tool known in the art may be used to analyze nucleic acids present in a sample and/or subject as described above, e.g., a spectrophotometer for absorption or calorimetric measurements, a fluorometer or flow cytometer for fluorescence measurements, a scintillation or gamma counter for radioactive measurements, and an automated cell counter, automated plate counter, or manual plate counter for cell number measurements. As a further example, a microwell reader can be used for fluorescence, absorbance or calorimetric measurements. In some embodiments, measurements to detect nucleic acids/nucleic acid amplification products may include analysis by digital PCR. In some embodiments, measurements to detect nucleic acids/nucleic acid amplification products may include analysis by droplet digital PCR using, for example, but not limited to, ddPCR™ using Bio-Rad's QX100™ or QX200™ Droplet Digital PCR systems. Measurement and analysis of amplification products may include analysis of 1D and/or 2D plots of fluorescence amplitude exhibited in a droplet digital PCR amplification.

### Identification

The present inventive concept further encompasses methods of identifying a fastidious microorganism as described herein, in a sample and/or a subject, for example, a sample derived from a subject that may be suspected of having an infection from a particular fastidious microorganism. The fastidious microorganism may be identified by detection of microbial nucleic acids, e.g., by direct sequencing, nucleic acid amplification, hybridization to probes, and the like, and analysis of the nucleic acids/nucleic acid amplification products detected. In some embodiments, identifying the fastidious microorganism may include nucleic acid amplification, for example, as described hereinabove, and detection of nucleic amplification products.

Methods of detecting nucleic acids are well-known in the art and may include specific hybridization of a fluorescently labeled or tagged probe to sequences characteristic for a fastidious microorganism and detecting fluorescence emission from the fluorescently tagged or labeled oligonucleotide probe. For example, nucleic acid amplification and/or primers may be used to identify particular characteristic nucleic acids and/or nucleic acid sequences to a particular fastidious microorganism. In some embodiments, these nucleic acids and/or nucleic acid sequences amplified and probed for are associated with, for example, the internal transcribed sequence (ITS) between 5S, 16S, and/or 23S rRNA genes, or associated with sequences within the 5S, 16S, and/or 23S rRNA genes of a particular fastidious microorganism. In some embodiments, the nucleic acid sequences amplified and probed for are associated with the ITS between the 16S and 23S rRNA genes. In other embodiments, the nucleic acid sequences amplified and probed for are associated with the ITS between the 23S and 5S rRNA genes. In some embodiments, identifying the fastidious microorganism may include specific hybridization to probe sequences and, for example, detecting fluorescence emission from a fluorescently tagged or labeled oligonucleotide probe that hybridizes to a nucleic acid and/or a nucleic acid amplification product and gives off/releases fluorescence emission as a result of hybridization with the nucleic acid and/or the nucleic acid amplification product.

In some embodiments, measurements to identify nucleic acids/nucleic acid amplification products may include analysis by digital PCR. In some embodiments, measurements to identify nucleic acids/nucleic acid amplification products may include analysis by droplet digital PCR using, for example, but not limited to, ddPCR™ using Bio-Rad's QX100™ or QX200™ Droplet Digital PCR systems. Measurement and analysis of amplification products may include analysis of 1D and/or 2D plots of fluorescence amplitude exhibited in a droplet digital PCR amplification. In some embodiments, a particular fastidious microorganism may exhibit a particular 2D fluorescence amplitude distribution in a droplet digital PCR amplification. As such, in some embodiments, a particular fastidious microorganism may be identified by comparison of the 2D fluorescence amplitude distribution exhibited for a sample against reference 2D fluorescence amplitude distributions from known samples including particular fastidious microorganisms. In some embodiments, amplification, detection, analysis and/or identification of nucleic acids/nucleic acid amplification products according to the inventive concept may be performed as described by Maggi et al., 2020. J. Microbiol. Methods. 176, 106022, incorporated herein by reference in its entirety for its disclosures.

### Enrichment

In some embodiments, methods of detecting and/or identifying fastidious microorganisms according to the present inventive concept may also include “enrichment” for the fastidious microorganism by, for example, culturing a sample, such as, a sample derived from a subject that may be suspected of having an infection from a particular fastidious microorganism, in an enrichment medium, prior to performing the methods for detecting and/or identifying fastidious microorganisms of the present inventive concept as described herein. In some embodiments, the sample derived from a subject may be cultured in an enrichment medium, for example, the culture medium as described in U.S. Pat. No. 7,115,385, hereby incorporated herein by reference in its entirety, prior to performing the methods for detecting and/or identifying fastidious microorganisms of the present inventive concept as described herein. For example, in some embodiments, the cell culture medium may include: free amino acids including glutamine at a concentration of about 300 to about 6000 mg/1; organic acid as a carbon source; hemin; yeast extract; nucleotides; vitamins; acyl homoserine lactone; and sodium bicarbonate, and wherein the culture medium is protein free. In some embodiments, the cell culture medium may be Bartonella a proteobacteria growth medium (BAPGM).

In other embodiments, the cell culture media for enrichment of the fastidious microorganism may include, for example, an insect and/or a mammalian cell culture media, however, is not limited thereto. In some embodiments, the cell culture media, for example, BAPGM, may further include any of one, any combination of, and/or all of the following supplements: β-Nicotinamide adenine dinucleotide (NAD); β-Nicotinamide adenine dinucleotide phosphate (NADPH); sodium pyruvate; adenosine 5′-triphosphate (ATP); essential amino acids; yeast extract; sodium bicarbonate; saponin; sodium polyanethol sulfonate (SPS); and/or serum (such as, but not limited to, fetal, horse, human, etc.), or any other supplement that would be appreciated by one of skill in the art. In some embodiments, the cell culture medium is protein-free.

In other embodiments, methods of detecting and/or identifying fastidious microorganisms according to the present inventive concept may include enrichment of, for example, biomarkers for a fastidious microorganism, such as particular characteristic nucleic acids and/or nucleic acid sequences to a particular fastidious microorganism by capture and/or concentration of characteristic nucleic acids and/or nucleic acid sequences to a particular fastidious microorganism. In some embodiments, these nucleic acids and/or nucleic acid sequences captured and concentrated are associated with, for example, the ITS between 5S, 16S, and/or 23S rRNA genes, or associated with sequences within the 5S, 16S, and/or 23S rRNA genes of a particular fastidious microorganism. In some embodiments, the nucleic acids and/or nucleic acid sequences captured and concentrated are associated with the ITS between the 16S and 23S rRNA genes. In other embodiments, the nucleic acid sequences captured and concentrated are associated with the ITS between the 23S and 5S rRNA genes. In some embodiments, the nucleic acids and/or nucleic acid sequences captured and concentrated may then further be amplified, detected, analyzed, and/or identified by methods for amplification, detection, and identification as described herein. In some embodiments, capturing and concentrating of nucleic acids and/or nucleic acid sequences may be accomplished, for example, using highly porous hydrogel particles, such as nanoparticles, functionalized with, for example, affinity baits, affinity groups, and/or affinity ligands which have very high affinities for the particular nucleic acids and/or nucleic acid sequences that are biomarkers for a particular fastidious microorganism.

In some embodiments, capturing and concentrating of nucleic acids and/or nucleic acid sequences using highly porous hydrogel particles can be accomplished using, for example, NANOTRAP® technology (Ceres Nanosciences, Inc. (Manassas, Va.)), including NANOTRAP® particles and/or NANOTRAP® magnetic particles, such as are described in, for example, U.S. Patent Application Publication Nos. 2009/0087346, 2009/0148961, 2012/0164749, and 2014/0045274. Nanoparticles, for example, NANOTRAP® particles and NANOTRAP® magnetic particles, functionalized with affinity baits, affinity groups, and/or affinity ligands that have very high affinities for the particular nucleic acids and/or nucleic acid sequences that are biomarkers for a particular fastidious microorganism may be used, for example, to capture the particular nucleic acids and/or nucleic acid sequences on the nanoparticles under conditions that the particular nucleic acids and/or nucleic acid sequences bind to/have high affinities for the nanoparticles, followed by separating/isolating the nanoparticles including the captured nucleic acids and/or nucleic acid sequences bound to the nanoparticles from the sample, and eluting the captured nucleic acids and/or nucleic acid sequences from the nanoparticles to provide an enrichment of/an enriched sample including the particular nucleic acids and/or nucleic acid sequences, such as those characteristic of a particular fastidious microorganism. The enriched sample may be suitable further analysis, such as detection and/or analysis of nucleic acids/nucleic acid sequences by the amplification, detection and identification methods described herein.

Having described various aspects of the present inventive concept, the same will be explained in further detail in the following examples, which are included herein for illustration purposes only, and which are not intended to be limiting to the inventive concept.

### Example 1

### Droplet Digital PCR Assay for the Detection and Quantification of Bartonella Species in Human Clinical Samples

Herein we present the development, optimization, and validation of a droplet digital PCR assay, using a QX200 Droplet Digital PCR (ddPCR™, Bio-Rad) system, for the detection of Bartonella spp. DNA within several sample matrices, including infected epithelial cell cultures and pre-characterized clinical blood samples. Our Bartonella spp. ddPCR assay was developed based upon previously published TaqMan-based qPCR assays that are able to detect over 25 Bartonella spp. [57-60], and includes host DNA (housekeeping gene) amplification as a reference target. The new Bartonella spp. ddPCR assay efficiency, sensitivity, and specificity was assessed by comparison with the current qPCR method used by the Intracellular Pathogens Research Laboratory (North Carolina State University, N.C., USA), and Galaxy Diagnostics (a clinical diagnostic company located within the Research Triangle Park, N.C., USA).

### Methods

### Sample Reference Types and DNA Extraction

Three matrices were tested to optimize the Bartonella spp ddPCR assay (see below): DNA from human blood samples spiked with Bartonella DNA, experimentally infected human cell lines, and pre-characterized clinical human blood samples. DNA extraction from all sample types was performed using either the Qiagen DNeasy Kit (tissue DNA extraction) or QIAsymphony DNA Kit (for blood and blood culture DNA extraction).

DNA Extracted from Human Blood Samples Spiked with Bartonella DNA

To assess the specificity of the Bartonella spp. ddPCR assay for detection of Bartonella spp. within human blood, DNA from 14 different Bartonella spp. and strains (Table 2) were spiked into DNA extracted from naïve (confirmed to be pathogen free via both PCR and IFA-based CLIA accredited diagnostic testing modalities at Galaxy Diagnostics) human blood. Blood DNA samples were spiked with Bartonella DNA at final concentrations ranging from 0.001 to 0.1 pg/ul (equivalent to 0.5 to 50 bacterial genome copies per microliter of blood). In addition, naïve human blood extracted DNA was spiked with different combinations of two Bartonella species (Table 2) in equal amounts (equivalent to 0.1 pg/ul each) to assess the capability of the Bartonella spp. ddPCR assay to detect and identify dual Bartonella spp. infections. Although the clinical relevance is unknown, infection with more than one Bartonella spp. has been reported in human clinical case reports [41, 57-59, 61-64].

### Experimentally Infected Cell Lines

In order to establish and optimize the amplification and detection of intracellular Bartonella DNA (in contrast to extracellular Bartonella DNA in blood, serum or liquid BAPGM culture medium) with the Bartonella spp. ddPCR assay, samples from experimentally infected human epithelial cells were assayed. MCF10A human epithelial mammary cells (kindly provided by Dr. Neil Spector, Duke University Department of Medicine and Duke Cancer Institute, Durham, N.C.) were infected in vitro at a multiplicity of infection (MOI) of 1:1; 10:1; 30:1 or 100:1 with B. henselae San Antonio 2 strain (BhSA2) for up to 72 hrs. Infected cells were collected every 24 hrs. to assess, by both qPCR and ddPCR, the DNA ratio of Bartonella/host cell (as bacterial genome copies per host cell). Briefly, cell culture wells of fully confluent epithelial cells were infected at the three MOIs with freshly collected B. henselae SA2 colonies and incubated at 35° C. with 100% relative humidity and 5% CO₂. Prior to each single-well DNA extraction, cells were treated with 150 ug/ml of gentamicin and incubated at 35° C. for 4 hrs, followed by 4 washes with sterile PBS. Cells were harvested by adding 300 μl of PBS and scraping with a sterile rubber spatula. Total DNA was extracted as described above. All experiments were performed in duplicate.

### Pre-Characterized Clinical Diagnostic Blood Samples

A set of 120 diagnostic blood samples obtained from Galaxy Diagnostics' biobank, defined as diagnostically “Negative” (absence of Bartonella DNA via ePCR® platform testing and lack of B. henselae and B. quintana IFA seroreactivity), and 112 samples, consisting of blood, or BAPGM enrichment blood cultures from 38 human clinical cases (tested through the ePCR® platform) from repositories maintained by the North Carolina State University Intracellular Pathogens Research Laboratory (IPRL) and Galaxy Diagnostics, were tested to compare the specificity and sensitivity of the Bartonella spp. ddPCR assay versus qPCR.

### Molecular Detection of Bartonella DNA

Quantitative PCR (qPCR) Analysis:

Quantitative PCR amplification of the Bartonella ITS region was performed as described previously [57-59], with minor modifications. Oligonucleotide primers BsppITS325s:

(Forward, SEQ ID NO: 1)
5′ CTTCAGATGATGATCCCAAGCCTTCTGGCG 3′
and
543as:
(Reverse, SEQ ID NO: 2)
5′ AATTGGTGGGCCTGGGAGGACTTG 3′
and
probe BsppITS500probe:
(SEQ ID NO: 3)
5′ FAM-GTTAGAGCGCGCGCTTGATAAG-IABkFQ 3′

were utilized for these assays (Table 3). Each 25 μl PCR reaction was prepared using 12.5 μl of SsoAdvanced™ Universal Probes Supermix (Bio-Rad, Hercules, Calif.), 0.2 μl of 100 μM of each forward primer, reverse primer, and TaqMan probe (IDT® DNA Technology, Coralville, Iowa), 7.5 μl of Ultra-Pure, molecular grade water (Genesee Scientific, San Diego, Calif., USA), and 5 μl of DNA from each sample tested. Quantitative PCR was performed in an CFX96® (Bio-Rad, Hercules, Calif.) under the following conditions: a single hot-start cycle at 95° C. for 3 minutes followed by 45 cycles of denaturing at 94° C. for 10 seconds, annealing at 68° C. for 10 seconds, and extension at 72° C. for 10 seconds. Amplification was completed by an additional cycle at 72° C. for 30 seconds. Positive amplification was assessed by analysis of detectable fluorescence vs cycle threshold values.
Droplet Digital PCR (ddPCR) Analysis:

Droplet digital PCR amplification of the Bartonella ITS region was conducted using the same primers and probes employed for qPCR, with minor modifications. ddPCR amplification of the housekeeping gene human hydroxymethylbilane synthase (FMBS) [65], was performed using the primers and probes listed in Table 3. Primers and probes for the HMBS housekeeping gene were validated independently (results not shown).

The 22 μl final ddPCR reaction contained 11 μl of ddPCR™ Supermix for probes (no dUTP) (Bio-Rad, Hercules, Calif.), 0.2 μl each of 100 μM forward and reverse primers, 0.2 μl of 100 μM probe (IDT® DNA Technology, Coralville, Iowa), 3.8 μl of molecular grade water, 5 μl of DNA from each sample tested, and 1 μl of HindIII DNA restriction enzyme. The ddPCR analysis was performed using a QX200 Droplet Digital PCR (Bio-Rad, Hercules, Calif.) system under the following conditions: a single hot-start cycle at 95° C. for 10 minutes, followed by 40 cycles of denaturing at 94° C. for 30 seconds, and annealing at 62.9° C. for 1 minute. A final extension at 98° C. was carried out for five minutes. Bio-Rad QuantaSoft Analysis Pro software was utilized to track and analyze the fluorescent drop distribution and positive detection threshold readings for each channel (FAM channel 1 for Bartonella, and HEX channel 2 for house-keeping gene amplification).

### Results

Bartonella Spp. DNA Spiked into DNA Extracted from Naïve Human Blood

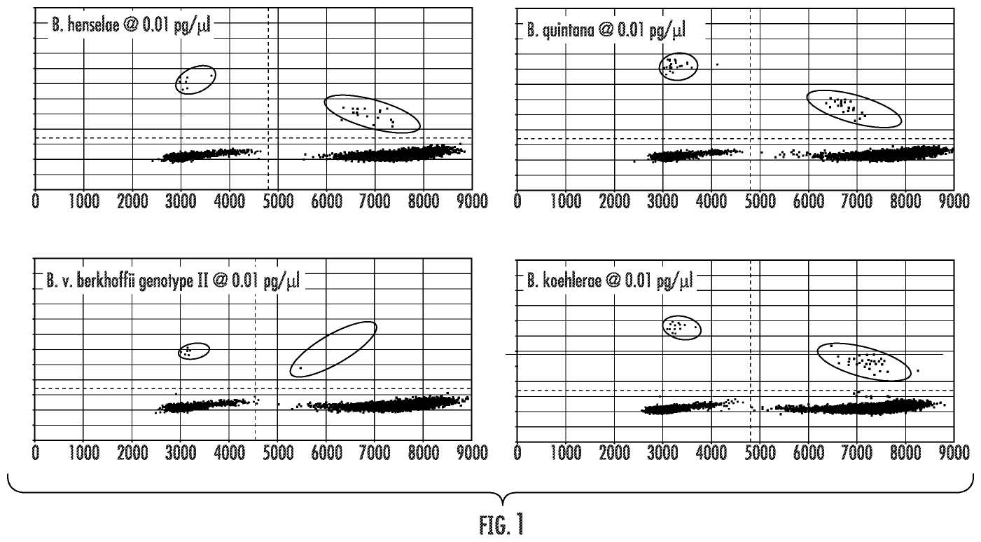

DNA extractions from B. henselae, B. quintana, B. koehlerae or B. vinsonii subsp. berkhoffii genotype II spiked into negative control human blood samples (at a concentration of 5 bacteria/μL) were used to set threshold values and determine the limit of detection (LOD) (FIG. 1). Bartonella spp. ddPCR assay parameters were successfully optimized to detect Bartonella concentrations equivalent to 0.5 bacterial genome copies per microliter of blood (Table 4). This is the same LOD previously quantified for Bartonella qPCR in our laboratories. The number of droplets detected (resolution) for each tested concentration was consistent across each of the four assessed time points (Table 4).

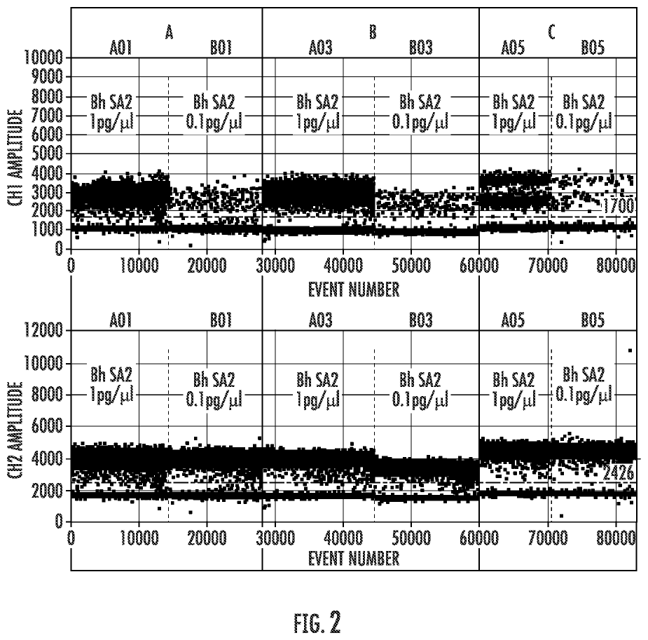

An important component of the ddPCR master mix is the addition of HindIII restriction enzyme to cut the DNA template into small fragments to facilitate more efficient DNA partitioning prior to amplification. The greatest DNA amplification and most sensitive signal resolution were obtained when HindIII was added to the premix, as compared with values obtained from either HindIII pre-digestion of DNA samples (HindIII incubated with DNA at 37° C. for 10 minutes prior to DNA addition to the premix as template), or in the absence of HindIII sample digestion (FIG. 2).

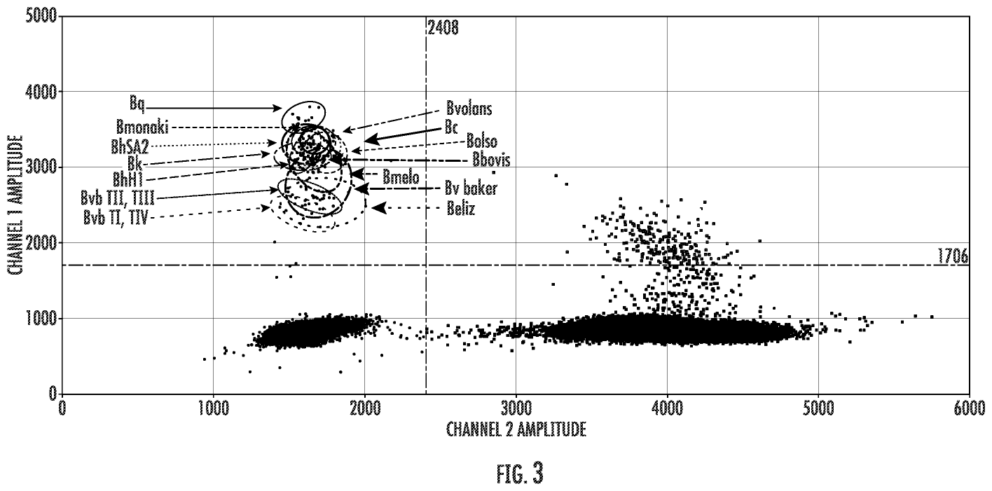

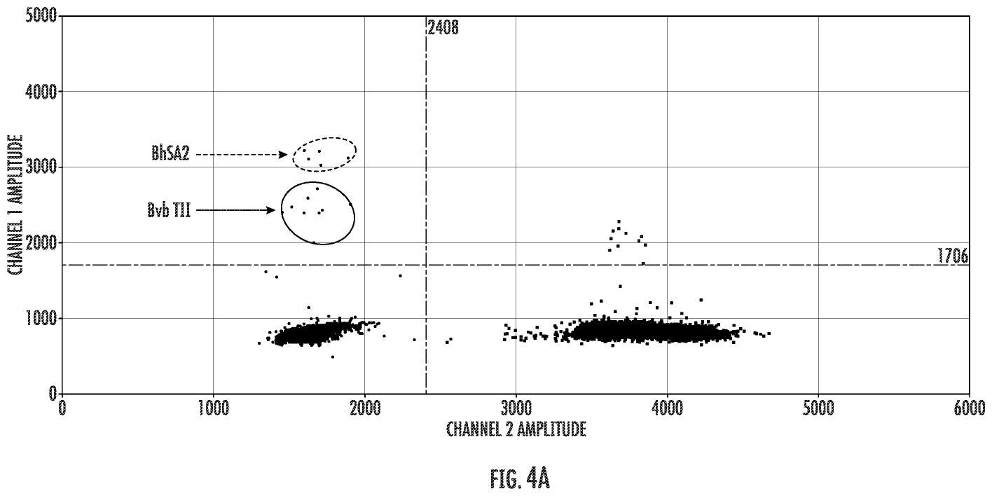

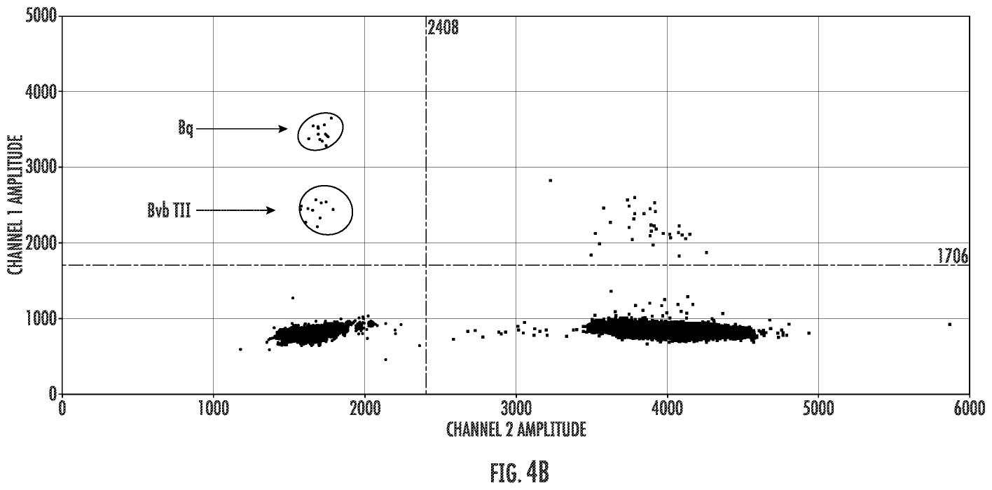

When DNA extractions from 14 different Bartonella spp. and strain isolates were singly spiked into naïve human blood DNA and when DNA from 2 clinical cases, infected with B. vinsonii berkhoffii genotype IV and B. alsatica (isolates not available in our collection), the Bartonella spp. ddPCR assay was able to detect all 16 species/strains: B. henselae Houston I (HI); B. henselae San Antonio 2 strain (SA2); B. quintana Fuller; B. vinsonii subsp. berkhoffii genotype I; B. vinsonii subsp. berkhoffii genotype II; B. vinsonii subsp. berkhoffii genotype III; B. vinsonii subsp. berkhoffii genotype IV*; B. vinsonii subsp. vinsonii; B. melophagi; B. volans; B. monaki; B. alsatica*; B. Bovis, B. elizabethae; B. clarridgeiae; B. koehlerae. (* species DNA extracted from naturally infected clinical cases). Bartonella spp. ddPCR assay amplification among this group included B. henselae, the etiologic agent of cat-scratch disease (CSD), as well as eight Bartonella spp. and strains associated with culture negative endocarditis in humans and dogs [19-30] (Table 2). Some species exhibited distinct, differential droplet distribution patterns (FIG. 3). Distinct droplet distribution signal patterns were also evident when dual species combinations were spiked at equal concentrations into naïve blood DNA (FIGS. 4A and 4B). The Bartonella spp. ddPCR assay detected all six different dual species combinations tested (Table 2). Importantly, the species-specific droplet distribution patterns obtained may facilitate the future simultaneous, single channel identification of multiple Bartonella spp. potentially present within a clinical sample, a feat not currently achievable with conventional or single-channel qPCR methodologies[66].

In-Vitro Bartonella henselae Infected Cell Lines

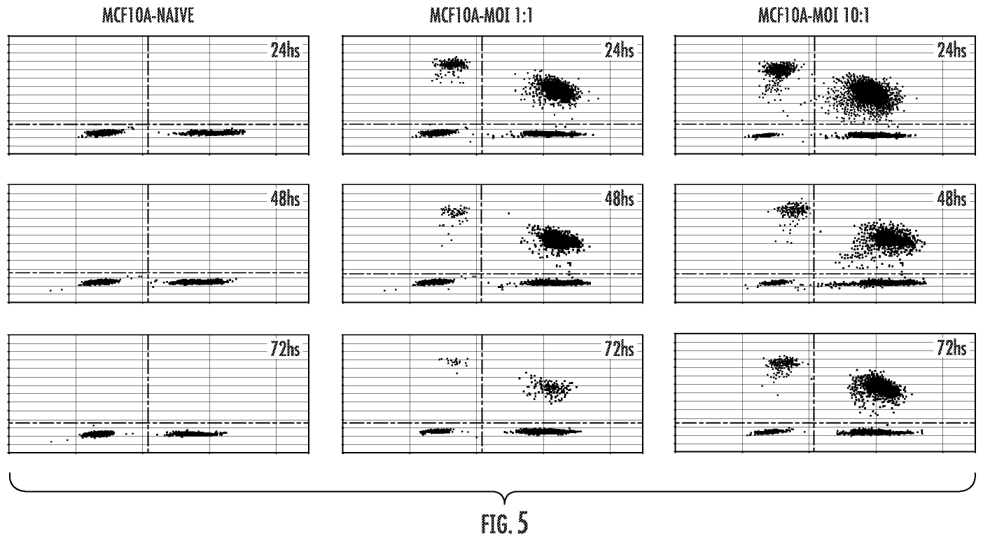

When MCF10A epithelial cells were infected in vitro with B. henselae SA2 strain, the intracellular ratio of Bartonella DNA content per host cell decreased over time, regardless of the MOI utilized (Table 5). The reduction of intracellular Bartonella DNA content as a function of time was also clearly demonstrated with ddPCR, as both droplet distribution intensity and number decreased over time (FIG. 5). For qPCR, calibration curves for each target gene (ITS for B. henselae and HMBS for host cell DNA) were obtained to assess DNA copy number compared with quantitation automatically calculated by software following ddPCR amplification. Results generated by ddPCR were in close agreement with those obtained with qPCR (results not shown).

### Pre-Characterized Clinical Samples

To assess specificity, 120 blood samples obtained from previously tested “Negative” clinical diagnostic accessions (absence of Bartonella DNA via ePCR® platform testing and lack of B. henselae and B. quintana IFA seroreactivity) were tested by ddPCR. When compared with the gold-standard ePCR® platform, Bartonella DNA was detected in only one sample that had been determined to be negative previously (n=119/120; 99% specificity). As both serology and qPCR can be negative in Bartonella spp. infected patients, this one sample could reflect a prior false negative result, or alternatively, a false positive ddPCR result.

When ddPCR sensitivity was evaluated by assaying 112 clinical samples consisting of blood, or BAPGM enrichment blood cultures incubated for either 7, 14, or 21 days, ddPCR testing resulted in the detection of six-fold more DNA positive results from blood DNA extractions, when compared with concurrent qPCR testing (Table 6).

Similarly, the ddPCR assay enhanced identification of Bartonella DNA positive samples following enrichment culture for 7, 14 or 21 days, as ddPCR detected first positives (i.e. the culture sample that provided first detection of Bartonella) at earlier culture time points when compared to qPCR (Table 6).

### Discussion and Conclusions

We present herein, the development and validation of a Droplet Digital PCR assay, using the QX200 (ddPCR™, Bio-Rad), that detects DNA of at least 16 Bartonella spp. and strains within spiked blood, cell culture-infected, and clinical human patient samples. We successfully adapted the previously reported TaqMan®-based qPCR assay to a ddPCR platform. This Bartonella spp. ddPCR assay proved to be a reliable means for detection and absolute quantification of DNA from Bartonella spiked blood DNA samples, exhibiting a sensitivity as low as 0.001 pg/ul of bacterial DNA. The assay also more efficiently detected Bartonella DNA within clinical samples, both directly from extracted blood samples and within 7-21 day enrichment blood cultures.

Our results highlight the advantages of ddPCR to significantly shorten the time needed to efficiently generate a sensitive, specific, and reliable Bartonella spp. positive DNA result, representing a clear advantage for the clinical management of bartonellosis patients. The development of a ddPCR assay for the detection and absolute quantification of target DNA in clinical samples has several advantages over conventional and quantitative PCR counterparts [67, 68]. Three of the most significant advantages are: the high resistance of ddPCR to different types of PCR amplification inhibitors, no need for inclusion of reference materials with known concentrations for ddPCR quantification, and increased precision of absolute target quantification at low template concentrations. Additionally, since the backbone of ddPCR is the production of thousands of discrete oil-water emulsion droplets to provide efficient sample partitioning, the additional, normally necessary, steps to avoid non-specific amplification and cross-contamination between droplets are minimized or eliminated [67].

The Bartonella spp ddPCR assay was able to detect not only Bartonella henselae, the etiological agent of cat-scratch disease (CSD), but also 8 of the 11 Bartonella spp. and strains associated with culture negative endocarditis in humans and dogs [19-30]: B. henselae, B. quintana, B. alsatica, B. ehzabethae, B. koehlerae, B. vinsonii subsp. berkhoffii genotype I, B. vinsonii subsp. berkhoffii genotype II, B. vinsonii subsp. berkhoffii genotype III, B. vinsonii subsp. berkhoffii genotype IV, B. vinsonii subsp. vinsonii, and B. clarridgeiae. Although CSD is a potentially self-limiting infection, culture negative endocarditis is a potentially life-threatening illness that demands diagnostic accuracy to facilitate directed therapy. In conclusion, the assay described in this report is the first step toward the development of a multiplex ddPCR assay (i.e. using the QX One from Bio-Rad) for the simultaneous detection and absolute quantification of multiple vector-borne pathogens (such as Babesia, Bartonella and Borrelia) within clinical samples.

### Example 1 References

1. Gutierrez-Aguirre, I., et al., Droplet digital PCR for absolute quantification of pathogens. Methods Mol Biol, 2015. 1302: p. 331-47.
2. Li, H., et al., Application of droplet digital PCR to detect the pathogens of infectious diseases. Biosci Rep, 2018. 38(6).
3. Sanders, R., et al., Evaluation of digital PCR for absolute DNA quantification. Anal Chem, 2011. 83(17): p. 6474-84.
4. Dong, L., et al., Evaluation of droplet digital PCR and next generation sequencing for characterizing DNA reference material for KRAS mutation detection. Sci Rep, 2018. 8(1): p. 9650.
5. King, J. L., et al., Validation of droplet digital PCR for the detection and absolute quantification of Borrelia DNA in Ixodes scapularis ticks. Parasitology, 2017. 144(4): p. 359-367.
6. Li, R., et al., [Detection of epidermal growth factor receptor gene mutations in different types of non-small cell lung cancer by droplet digital PCR and amplification refractory mutation system]. Zhonghua Bing Li Xue Za Zhi, 2017. 46(11): p. 764-768.
7. Srisutham, S., et al., Four human Plasmodium species quantification using droplet digital PCR. PLoS One, 2017. 12(4): p. e0175771.
8. Taylor, S. C., et al., Optimization of Droplet Digital PCR from RNA and DNA extracts with direct comparison to RT-qPCR: Clinical implications for quantification of Oseltamivir-resistant subpopulations. J Virol Methods, 2015. 224: p. 58-66.
9. Taylor, S. C., G. Laperriere, and H. Germain, Droplet Digital PCR versus qPCR for gene expression analysis with low abundant targets: from variable nonsense to publication quality data. Sci Rep, 2017. 7(1): p. 2409.
10. Tong, Y., et al., Application of Digital PCR in Detecting Human Diseases Associated Gene Mutation. Cell Physiol Biochem, 2017. 43(4): p. 1718-1730.
11. Wilson, M., et al., Development of droplet digital PCR for the detection of Babesia microti and Babesia duncani. Exp Parasitol, 2015. 149: p. 24-31.
12. Wu, X., et al., Development and application of a reverse transcriptase droplet digital PCR (RT-ddPCR)for sensitive and rapid detection of Japanese encephalitis virus. J Virol Methods, 2017.
13. Zhang, R., et al., Diagnostic accuracy of droplet digital PCR for detection of EGFR T790M mutation in circulating tumor DNA. Cancer Manag Res, 2018. 10: p. 1209-1218.
14. Sanders, R., et al., Evaluation of digital PCR for absolute RNA quantification. PLoS One, 2013. 8(9): p. e75296.
15. Dong, L., et al., Comparison of four digital PCR platforms for accurate quantification of DNA copy number of a certified plasmid DNA reference material. Sci Rep, 2015. 5: p. 13174.
16. Bessetti, J. An Introduction to PCR Inhibitors. Promega Corporation. 2007. 2.
17. Alaeddini, R., Forensic implications of PCR inhibition—A review. Forensic Sci Int Genet, 2012. 6(3): p. 297-305.
18. Dingle, T. C., et al., Tolerance of droplet-digital PCR vs real-time quantitative PCR to inhibitory substances. Clin Chem, 2013. 59(11): p. 1670-2.
19. von Loewenich, F. D., et al., Prosthetic Valve Endocarditis with Bartonella washoensis in a Human European Patient and its Detection in Red Squirrels (Sciurus vulgaris). J Clin Microbiol, 2019.
20. Kuncova, K., et al., Diagnosis and treatment of Bartonella endocarditis. Epidemiol Mikrobiol Imunol, 2019. 68(2): p. 104-108.
21. Andre, M. R., et al., Aortic valve endocarditis due to Bartonella clarridgeiae in a dog in Brazil. Rev Bras Parasitol Vet, 2019.
22. Tabar, M. D., et al., First description of Bartonella koehlerae infection in a Spanish dog with infective endocarditis. Parasit Vectors, 2017. 10(1): p. 247.
23. Shelnutt, L. M., et al., Death of Military Working Dogs Due to Bartonella vinsonii Subspecies berkhoffii Genotype III Endocarditis and Myocarditis. Mil Med, 2017. 182(3): p. e1864-e1869.
24. Penafiel-Sam, J., et al., Infective endocarditis due to Bartonella bacilliformis associated with systemic vasculitis: a case report. Rev Soc Bras Med Trop, 2017. 50(5): p. 706-708.
25. Okaro, U., et al., Bartonella Species, an Emerging Cause of Blood-Culture-Negative Endocarditis. Clin Microbiol Rev, 2017. 30(3): p. 709-746.
26. Ghashghaei, R., et al., Bartonella endocarditis. J Cardiol Cases, 2016. 13(1): p. 1-3.
27. Ohad, D. G., et al., Molecular detection of Bartonella henselae and Bartonella koehlerae from aortic valves of Boxer dogs with infective endocarditis. Vet Microbiol, 2010. 141(1-2): p. 182-5.
28. Lin, E. Y., et al., Candidatus Bartonella mayotimonensis and endocarditis. Emerg Infect Dis, 2010. 16(3): p. 500-3.
29. Maillard, R., et al., Endocarditis in cattle caused by Bartonella bovis. Emerg Infect Dis, 2007. 13(9): p. 1383-5.
30. Weinborn-Astudillo, R. M., et al., Bartonella Infection in Stray Dogs from Central and Southern Chile (Linares and Puerto Montt). Vector Borne Zoonotic Dis, 2019.
31. Chondrogiannis, K., et al., Seronegative cat-scratch disease diagnosed by PCR detection of Bartonella henselae DNA in lymph node samples. Braz J Infect Dis, 2012. 16(1): p. 96-9.
32. Weinspach, S., et al., Cat scratch disease—heterogeneous in clinical presentation: five unusual cases of an infection caused by Bartonella henselae. Klin Padiatr, 2010. 222(2): p. 73-8.
33. Boillat, N. and G. Greub, [Cat scratch disease and other human infections caused by Bartonella species]. Rev Med Suisse, 2008. 4(152): p. 901-7.
34. Corral, J., et al., First Report of Bacillary Angiomatosis by Bartonella elizabethae in an HIV-Positive Patient. Am J Dermatopathol, 2019. 41(10): p. 750-753.
35. Orsag, J., et al., Cutaneous bacillary angiomatosis due to Bartonella quintana in a renal transplant recipient. Transpl Int, 2015. 28(5): p. 626-31.
36. Mitchell, B. M. and R. L. Font, Molecular detection of Bartonella henselae for the diagnosis of cat scratch disease and bacillary angiomatosis of the conjunctiva. Cornea, 2011. 30(7): p. 807-14.
37. Wong, R., J. Tappero, and C. J. Cockerell, Bacillary angiomatosis and other Bartonella species infections. Semin Cutan Med Surg, 1997. 16(3): p. 188-99.
38. Breitschwerdt, E. B., et al., Bartonella henselae Bloodstream Infection in a Boy With Pediatric Acute-Onset Neuropsychiatric Syndrome. J Cent New Syst Dis, 2019. 11: p. 1179573519832014.
39. Mascarelli, P. E., et al., Bartonella henselae infection in a family experiencing neurological and neurocognitive abnormalities after woodlouse hunter spider bites. Parasit Vectors, 2013. 6: p. 98.
40. Breitschwerdt, E. B., et al., Hallucinations, sensory neuropathy, and peripheral visual deficits in a young woman infected with Bartonella koehlerae. J Clin Microbiol, 2011. 49(9): p. 3415-7.
41. Breitschwerdt, E. B., et al., Bartonella vinsonii subsp. berkhoffii and Bartonella henselae bacteremia in a father and daughter with neurological disease. Parasit Vectors, 2010. 3(1): p. 29.
42. Andersson, S. G. and V. A. Kempf, Host cell modulation by human, animal and plant pathogens. Int J Med Microbiol, 2004. 293(7-8): p. 463-70.
43. Franz, B. and V. A. Kempf, Adhesion and host cell modulation: critical pathogenicity determinants of Bartonella henselae. Parasit Vectors, 2011. 4: p. 54.
44. Arvand, M., et al., Bartonella henselae-specific cell-mediated immune responses display a predominantly Th1 phenotype in experimentally infected C57BL/6 mice. Infect Immun, 2001. 69(10): p. 6427-33.
45. Il'ina, T. S. and V. N. Bashkirov, [Interaction of bacteria of the genus Bartonella with the host: inhibition of apoptosis, induction of proliferation, and formation of tumors]. Mol Gen Mikrobiol Virusol, 2008(3): p. 3-11.
46. Pulliainen, A. T. and C. Dehio, Bartonella henselae: subversion of vascular endothelial cellfunctions by translocated bacterial effector proteins. Int J Biochem Cell Biol, 2009. 41(3): p. 507-10.
47. Pulliainen, A. T. and C. Dehio, Persistence of Bartonella spp. stealth pathogens: from subclinical infections to vasoproliferative tumor formation. FEMS Microbiol Rev, 2012. 36(3): p. 563-99.
48. Resto-Ruiz, S., A. Burgess, and B. E. Anderson, The role of the host immune response in pathogenesis of Bartonella henselae. DNA Cell Biol, 2003. 22(6): p. 431-40.
49. Chomel, B. B., et al., Ecological fitness and strategies of adaptation of Bartonella species to their hosts and vectors. Vet Res, 2009. 40(2): p. 29.
50. Maggi, R. G., A. W. Duncan, and E. B. Breitschwerdt, Novel chemically modified liquid medium that will support the growth of seven hartonella species. J Clin Microbiol, 2005. 43(6): p. 2651-5.
51. Hong, J., et al., Lymphatic Circulation Disseminates Bartonella Infection Into Bloodstream. J Infect Dis, 2017. 215(2): p. 303-311.
52. Wolf, A. L. N. A. C. R. G. M. E. B. B., In Pursuit of a Stealth Pathogen: Laboratory Diagnosis of Bartonellosis. Clinical Microbiology Newsletter, 2014. 36(5): p. 33-38.
53. Harms, A. and C. Dehio, Intruders below the radar: molecular pathogenesis of Bartonella spp. Clin Microbiol Rev, 2012. 25(1): p. 42-78.
54. Lynch, T., J. Iverson, and M. Kosoy, Combining culture techniques for Bartonella: the best of both worlds. J Clin Microbiol, 2011. 49(4): p. 1363-8.
55. Riess, T., et al., Analysis of a novel insect-cell culture medium-based growth medium for Bartonella species. Appl Environ Microbiol, 2008.
56. Weeden, A. L., et al., Bartonella henselae in canine cavitary effusions: prevalence, identification, and clinical associations. Vet Clin Pathol, 2017. 46(2): p. 326-330.
57. Breitschwerdt, E. B., et al., Bartonella spp. Bloodstream Infection in a Canadian Family. Vector Borne Zoonotic Dis, 2019. 19(4): p. 234-241.
58. Breitschwerdt, E. B. and R. G. Maggi, Bartonella quintana and Bartonella vinsonii subsp. vinsonii bloodstream co-infection in a girl from North Carolina, USA. Med Microbiol Immunol, 2019. 208(1): p. 101-107.
59. Oteo, I A., et al., Prevalence of Bartonella spp. by culture, PCR and serology, in veterinary personnel from Spain. Parasit Vectors, 2017. 10(1): p. 553.
60. Messinger, C I, et al., Seroprevalence of Bartonella species, Coxiella burnetii and Toxoplasma gondii among patients with hematological malignancies: A pilot study in Romania. Zoonoses Public Health, 2017. 64(6): p. 485-490.
61. Breitschwerdt, E. B., et al., Bartonella species in blood of immunocompetent persons with animal and arthropod contact. Emerg Infect Dis, 2007. 13(6): p. 938-41.
62. Breitschwerdt, E. B., et al., Molecular evidence of perinatal transmission of Bartonella vinsonii subsp. berkhoffii and Bartonella henselae to a child. J Clin Microbiol, 2010. 48(6): p. 2289-93.
63. Breitschwerdt, E. B., et al., Bartonella sp. bacteremia in patients with neurological and neurocognitive dysfunction. J Clin Microbiol, 2008. 46(9): p. 2856-61.
64. Maggi, R. G., et al., Bartonella spp. bacteremia in high-risk immunocompetent patients. Diagn Microbiol Infect Dis, 2011. 71(4): p. 430-7.
65. Cicinnati, V. R., et al., Validation of putative reference genes for gene expression studies in human hepatocellular carcinoma using real-time quantitative RT-PCR. BMC Cancer, 2008. 8: p. 350.
66. Maggi, R. G. and E. B. Breitschwerdt, Potential limitations of the 16S-23S rRNA intergenic region for molecular detection of Bartonella species. J Clin Microbiol, 2005. 43(3): p. 1171-6.
67. Cao, L., et al., Advances in digital polymerase chain reaction (dPCR) and its emerging biomedical applications. Biosens Bioelectron, 2017. 90: p. 459-474.
68. Pinheiro-de-Oliveira, T. F., et al., Reverse transcriptase droplet digital PCR to identify the emerging vesicular virus Senecavirus A in biological samples. Transbound Emerg Dis, 2019. 66(3): p. 1360-1369.

### Example 2

### Multiplexed Droplet Digital PCR Assay for the Detection and Quantification of Bartonella and Borrelia Species

This report details the use of the Bio-Rad QX200 Droplet Digital (ddPCR) PCR system for the development, optimization, and validation of a Bartonella and Borrelia species multiplex ddPCR (BBddPCR) assay for pathogen detection within two different matrices—an experimentally infected histiocytic cell line and spiked blood samples. The BBddPCR assay presented herein is based upon the Bartonella spp. ddPCR assay (See, EXAMPLE 1). Both the Borrelia species ddPCR and host house-keeping gene assays were developed and validated, as described below.

### Methods

### Sample Reference Types and DNA Extraction

Two matrices were tested to optimize the BBddPCR assay (see below): DNA from in vitro canine histiocytic cell (DH82) cultures experimentally infected with Borrelia burgdorferi, and DNA from canine or human blood samples spiked with Bartonella and/or Borrelia spp. DNA. DNA extraction from all samples types was performed using either the Zymo Research Quick-DNA/RNA Miniprep Kit (for DH82 cellular DNA extraction) or the QIAsymphony DNA Kit (for blood DNA extraction).

### Intracellular Infection of In Vitro Cultivated Mammalian Cell Lines

ddPCR detection of intracellular Bartonella spp DNA within experimentally infected mammalian cell cultures was described elsewhere (See, EXAMPLE 1).

For intracellular detection of Borrelia DNA, triplicate wells of confluent DH82 cells were infected in vitro with B. burgdorferi clonal strain B31-MI 16 strain at a multiplicity of infection (MOI) of 10:1 and incubated at 37° C. with 5% CO₂ for 24 to 72 hours. Prior to DNA extraction, cells were treated with 150 ug/ml gentamicin for 4 hrs at 37° C. with 5% CO₂ and gently washed three times with sterile PBS. Cells were harvested from each well via pipetting up and down following the addition of 1 ml of ice-cold PBS-5 mM EDTA and a 15-minute incubation at 4° C. Cellular DNA was extracted, as described above, at 24, 48, and 72 hrs post infection. BBddPCR was then conducted as described below.

DNA Extracted from Spiked Blood Samples

In order to optimize the amplification and detection of intracellular Borrelia DNA, blood samples from naïve dogs were spiked with B. burgdorferi strain B31 DNA at final concentrations of 1; 0.1; 0.01; or 0.001 pg/ul). To assess the specificity of the BBddPCR assay for the detection of Bartonella and Borrelia spp DNA within a single matrix, Bartonella spp. DNA from 3 different species (B. henselae SA2, B quintana, or B. vinsonii berkhoffii genotype II, Borrelia burgdorferi B31 DNA, or a combination of B. burgdorferi+Bartonella spp DNA, were each spiked into naïve human blood at a final concentration of 0.1 pg/ul. Blood DNA was extracted from each sample as described above, and BBddPCR was conducted as mentioned below.

### Molecular Methods

ddPCR Analysis of Borrelia and House-Keeping Gene DNA:

For DH82 cell line infection testing, Borrelia ddPCR detection was performed using oligonucleotides BobuITS120s: 5′ AGGTCATTTTGGGGGTTTAGCTCAGTTGGCT 3′ (SEQ ID NO:4) and BoLymeITS200as-m 5′ AATGGAGGTTAAGGGACTCGAACCCT 3′ (SEQ ID NO:5) aimed at a 104 bp segment of the intergenic region (ITS), as forward and reverse primers, respectively. Oligonucleotide BobuITS160: 5′ FAM-CGGCTTTGCAAGCCGAGGGTCAAG-IABkFQ 3′ (SEQ ID NO:6) was used as the Taqman probe for Borrelia detection in channel 1. Similarly, oligonucleotides CaFeBRAF-15s: 5′ TCAYGAAGACCTCACAGTAAAAATAGGT 3′ (SEQ ID NO:7) and CaFeBRAF-110as: 5′ GATCCAGACAACTGTTCAAACTGATG 3′ (SEQ ID NO:8) were used as forward and reverse primers for the detection of a 95 bp segment of the Braf gene (used as a house-keeping gene) [70, 71]. Detection of Braf amplification was accomplished by including the CaFeBRAF-50 probe: 5′ HEX-GTCTAGCCACAGTGAAATCTCGATG-BHQ-2 3′ (SEQ ID NO:9) as a fluorescent probe for detection in channel 2.

The ddPCR reaction consisted of a 22-μl final PCR reaction consisting of: 11 μl of ddPCR™ Supermix for probes (no dUTP) (Bio-Rad, Hercules, Calif.), 0.2 μl of 100 μM of each Taqman probe, forward and reverse primer, (IDT® DNA Technology, Coralville, Iowa), 3.8 μl of molecular-grade water, 5 μl of sample DNA, and 1 μl of HindIII DNA restriction enzyme. The ddPCR analysis was performed using a QX200 Droplet Digital PCR (Bio-Rad, Hercules, Calif.) system under the following conditions: a single hot-start cycle at 95° C. for 10 minutes followed by 40 cycles of denaturing at 94° C. for 30 seconds and annealing at 62.9° C. for 1 minute. A final extension at 98° C. was carried for five minutes. Fluorescent droplet detection and distribution readings were recorded in channel 1 (for Borrelia DNA) and channel 2 (for house-keeping DNA).

ddPCR Analysis of Bartonella and Borrelia DNA:

ddPCR detection of Bartonella DNA, aimed at a 200 bp (depending on species) segment of the intergenic region (ITS), was performed as described previously, with minor modifications (See, EXAMPLE 1). Briefly, oligonucleotides BsppITS325s: 5′ CTTCAGATGATGATCCCAAGCCTTCTGGCG 3′ (SEQ ID NO:1) and BsppITS543as: 5′ AATTGGTGGGCCTGGGAGGACTTG 3′ (SEQ ID NO:2) were used as forward and reverse primers, respectively, while oligonucleotide BsppITS500 probe: 5′ FAM-GTTAGAGCGCGCGCTTGATAAG-IABkFQ 3′ (SEQ ID NO:3) was used as the taqman probe for Bartonella detection (Channel 1)

Borrelia ddPCR detection was performed using the same primer set described above, and the probe BobuITS160: 5′ HEX-CGGCTTTGCAAGCCGAGGGTCAAG-BHQ-2 3′ (SEQ ID NO:10). This probe has the same sequence as the one described above but utilizes the HEX fluorescent dye instead of FAM to facilitate Borrelia detection in channel 2.

The reaction preparation and cycling conditions described above were also used for the simultaneous detection of Bartonella and Borrelia DNA. Fluorescent droplet detection and distribution readings were recorded in channel 1 (for Bartonella DNA) and channel 2 (for Borrelia DNA).

### Results

### Intracellular Infection of In Vitro Cultivated Mammalian Cells

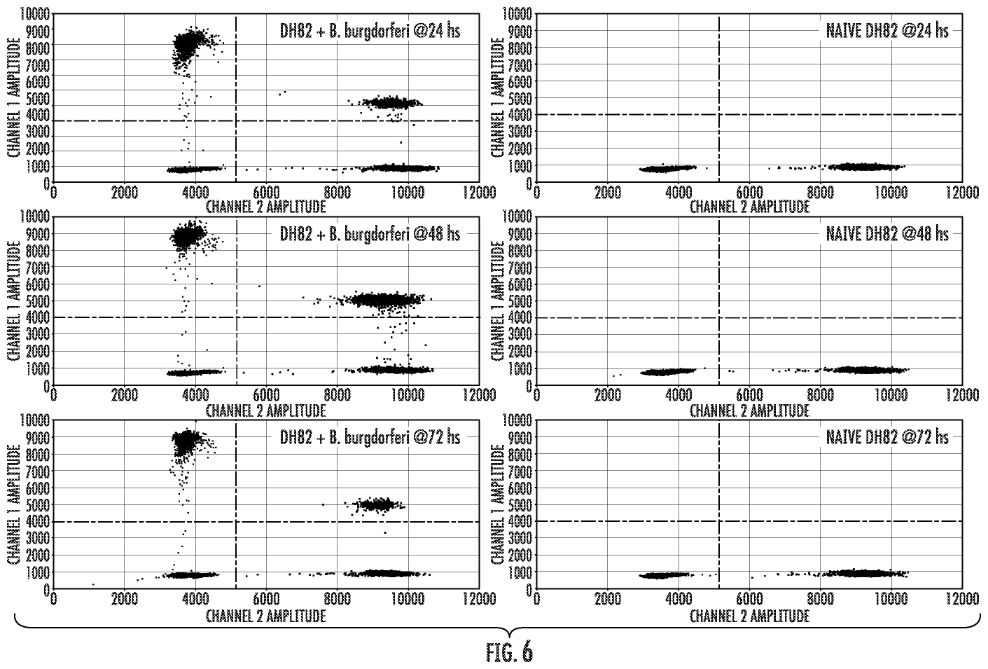

Amplification and detection of (within DH82 cells) Borrelia DNA was observed using channel 1 within all intracellular fractions (FIG. 6), but not within naïve (un-infected) DH82 cells processed in an identical manner, and at the same time. Borrelia DNA was detected at 24, 48, and 72 hrs post infection (FIG. 6), but was not detected within naïve DH82 cells or cells infected with Bartonella henselae SA2. Only the house-keeping gene was amplified from these groups of cells (channel 2).

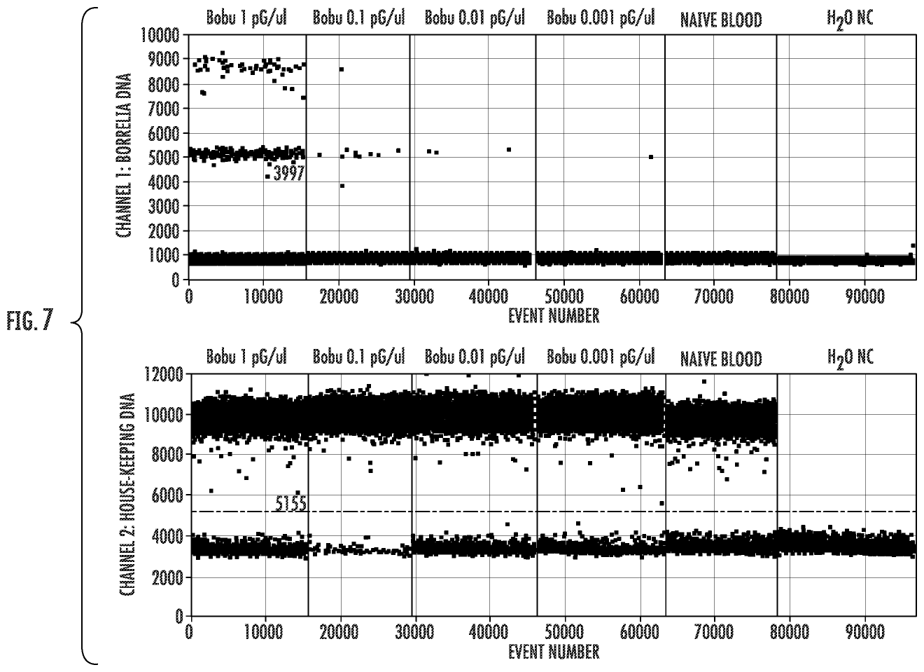

Similarly, Borrelia DNA was detected at concentrations of 1; 0.1; 0.01; and 0.001 pg/ul when spiked into dog blood, but was not detected in naïve blood DNA samples, or when water was used as the template (FIG. 7).

Multiplex Bartonella and Borrelia ddPCR Assay (BBddPCR)
Spiked naïve blood samples

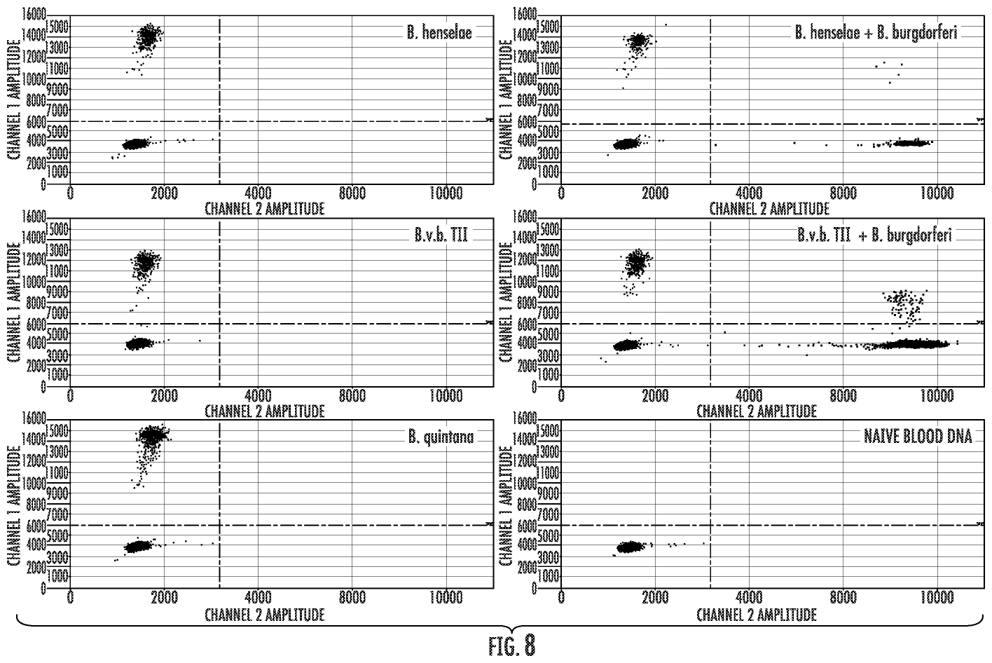

Amplification and detection of Borrelia and Bartonella DNA was observed for all species (B. burgdorferi B31, B. henselae, B. quintana and B. v. berkhoffii genotype II) in each respective channel when DNA of each target organism was spiked into naïve human blood samples (FIG. 8).

The ddPCR conditions used in this BBddPCR assay resulted in excellent droplet detection and signal resolution (see circled drop signals) for the detection of both Bartonella and Borrelia spp DNA at concentrations equivalent to 5 bacteria genome copies per microliter of blood. Previous work showed that this assay detects down to 0.001 pg/ul (equivalent to 0.5 bacteria per microliter) for all three Bartonella species tested (See, EXAMPLE 1).

No signal due to detection of Borrelia DNA was observed (channel 2) when naïve human blood was spiked with any of the three Bartonella species tested, and no detection of Bartonella DNA was observed (channel 1) when naïve human blood was spiked only with B. burgdorferi DNA (FIG. 3). Similarly, signals from the amplification of both pathogens were detected only when a combination of Bartonella (regardless the species) and Borrelia DNA (mimicking dual-infection) was tested. Pathogen specific amplification signals were not detected in naïve human blood DNA (FIG. 3).

Similar results were obtained when DNA from B. henselae, B. quintana, B. v. berkhoffii genotype II, and B. burgdorferi were spiked in the same manner into naive dog blood (results not shown).

### CONCLUSIONS

We presented the development, evaluation, and validation of a Droplet Digital PCR assay, the BBddPCR assay, using the QX200 (ddPCR™, Bio-Rad), for the multiplex detection of Bartonella and Borrelia spp DNA in human and animal samples. This ddPCR assay is also able to detect B. burgdorferi and B. bissettii present within naturally infected tick samples [72], as well as 16 Bartonella species and strains (results not shown) at concentrations ranging from 0.001 pg/ul to 0.01 pg/ul (See, EXAMPLE 1).

The assay presented here detects two Borrelia species associated with human borreliosis: B. burgdorferi and B. bissettii. Further testing will be aimed at the detection of other Borrelia species (such as B. garinii, B. mayoni, and B. afzelii) associated with human borreliosis.

### Example 2 References

1. Zhang, R., et al., Diagnostic accuracy of droplet digital PCR for detection of EGFR T790M mutation in circulating tumor DNA. Cancer Manag Res, 2018. 10: p. 1209-1218.
2. Kim, S. S., et al., Droplet digital PCR-based EGFR mutation detection with an internal quality control index to determine the quality of DNA. Sci Rep, 2018. 8(1): p. 543.
3. Dong, L., et al., Evaluation of droplet digital PCR and next generation sequencing for characterizing DNA reference material for KRAS mutation detection. Sci Rep, 2018. 8(1): p. 9650.
4. Zhang, Y., et al., Total DNA input is a crucial determinant of the sensitivity of plasma cell-free DNA EGFR mutation detection using droplet digital PCR. Oncotarget, 2017. 8(4): p. 5861-5873.
5. Tong, Y., et al., Application of Digital PCR in Detecting Human Diseases Associated Gene Mutation. Cell Physiol Biochem, 2017. 43(4): p. 1718-1730.
6. Millier, M. J., L. K. Stamp, and P. A. Hessian, Digital-PCR for gene expression: impact from inherent tissue RNA degradation. Sci Rep, 2017. 7(1): p. 17235.
7. Li, R., et al., [Detection of epidermal growth factor receptor gene mutations in different types of non-small cell lung cancer by droplet digital PCR and amplification refractory mutation system]. Zhonghua Bing Li Xue Za Zhi, 2017. 46(11): p. 764-768.
8. Taylor, S. C., et al., Optimization of Droplet Digital PCR from RNA and DNA extracts with direct comparison to RT-qPCR: Clinical implications for quantification of Oseltamivir-resistant subpopulations. J Virol Methods, 2015. 224: p. 58-66.
9. Li, H., et al., Application of droplet digital PCR to detect the pathogens of infectious diseases. Biosci Rep, 2018. 38(6).
10. Srisutham, S., et al., Four human Plasmodium species quantification using droplet digital PCR. PLoS One, 2017. 12(4): p. e0175771.
11. Luo, J., et al., Accurate detection of methicillin-resistant Staphylococcus aureus in mixtures utilizing single bacterial duplex droplet digital PCR. J Clin Microbiol, 2017.
12. King, J. L., et al., Validation of droplet digital PCR for the detection and absolute quantification of Borrelia DNA in Ixodes scapularis ticks. Parasitology, 2017. 144(4): p. 359-367.
13. Wilson, M., et al., Development of droplet digital PCR for the detection of Babesia microti and Babesia duncani. Exp Parasitol, 2015. 149: p. 24-31.
14. Gutierrez-Aguirre, I., et al., Droplet digital PCR for absolute quantification of pathogens. Methods Mol Biol, 2015. 1302: p. 331-47.
15. Bian, X., et al., A microfluidic droplet digital PCR for simultaneous detection of pathogenic Escherichia coli 0157 and Listeria monocytogenes. Biosens Bioelectron, 2015. 74: p. 770-7.
16. Ruelle, J., et al., Validation of an ultrasensitive digital droplet PCR assay for HIV-2plasma RNA quantification. J Int AIDS Soc, 2014. 17(4 Suppl 3): p. 19675.
17. Bessetti, J. An Introduction to PCR Inhibitors. Promega Corporation. 2007. 2.
18. Alaeddini, R., Forensic implications of PCR inhibition—A review. Forensic Sci Int Genet, 2012. 6(3): p. 297-305.
19. Dingle, T. C., et al., Tolerance of droplet-digital PCR vs real-time quantitative PCR to inhibitory substances. Clin Chem, 2013. 59(11): p. 1670-2.
20. Paddock, C., et al. Global Health Impacts of Vector Borne Diseases: Changing paradigms for tick-borne diseases in the Americas. in Forum on Microbial Threats; Board on Global Health; Health and Medicine Division; National Academies of Sciences, Engineering, and Medicine. 2016. Washington (DC): National Academies Press.
21. Mead, P. S., Updated CDC Recommendation for Serologic Diagnosis of Lyme Disease, C.f.D.C.a. Prevention, Editor 2019, Center for Disease Control and Prevention: Center for Disease Control and Prevention. p. 703.
22. Lantos, P. M., Chronic Lyme disease. Infect Dis Clin North Am, 2015. 29(2): p. 325-40.
23. Lantos, P. M. and G. P. Wormser, Chronic coinfections in patients diagnosed with chronic Lyme disease: a systematic review. Am J Med, 2014. 127(11): p. 1105-10.
24. CDC. https://www.niaid.nih.gov/diseases-conditions/lyme-disease-co-infection.
25. Christmann, D., [Lyme borreliosis and co-infections. Place of Anaplasma phagocytophilum and Bartonella henselae]. Bull Acad Natl Med, 2015. 199(4-5): p. 617-26; discussion 626-8.
26. Horowitz, R. I. and P. R. Freeman, Precision medicine: retrospective chart review and data analysis of 200 patients on dapsone combination therapy for chronic Lyme disease/post-treatment Lyme disease syndrome: part J. Int J Gen Med, 2019. 12: p. 101-119.
27. Johnson, L., et al., Severity of chronic Lyme disease compared to other chronic conditions: a quality of life survey. PeerJ, 2014. 2: p. e322.
28. Mayne, P. J., Emerging incidence of Lyme borreliosis, babesiosis, bartonellosis, and granulocytic ehrlichiosis in Australia. Int J Gen Med, 2011. 4: p. 845-52.
29. Mayne, P. J., Clinical determinants of Lyme borreliosis, babesiosis, bartonellosis, anaplasmosis, and ehrlichiosis in an Australian cohort. Int J Gen Med, 2015. 8: p. 15-26.
30. Maggi, R. G., et al., Bartonella spp. bacteremia and rheumatic symptoms in patients from Lyme disease-endemic region. Emerg Infect Dis, 2012. 18(5): p. 783-91.
31. Kuncova, K., et al., Diagnosis and treatment of Bartonella endocarditis. Epidemiol Mikrobiol Imunol, 2019. 68(2): p. 104-108.
32. Penafiel-Sam, J., et al., Infective endocarditis due to Bartonella bacilliformis associated with systemic vasculitis: a case report. Rev Soc Bras Med Trop, 2017. 50(5): p. 706-708.
33. Okaro, U., et al., Bartonella Species, an Emerging Cause of Blood-Culture-Negative Endocarditis. Clin Microbiol Rev, 2017. 30(3): p. 709-746.
34. Breitschwerdt, E. B., Bartonellosis, One Health and all creatures great and small. Vet Dermatol, 2017. 28(1): p. 96-e21.
35. von Loewenich, F. D., et al., Prosthetic Valve Endocarditis with Bartonella washoensis in a Human European Patient and its Detection in Red Squirrels (Sciurus vulgaris). J Clin Microbiol, 2019.
36. Andre, M. R., et al., Aortic valve endocarditis due to Bartonella clarridgeiae in a dog in Brazil. Rev Bras Parasitol Vet, 2019.
37. Tabar, M. D., et al., First description of Bartonella koehlerae infection in a Spanish dog with infective endocarditis. Parasit Vectors, 2017. 10(1): p. 247.
38. Shelnutt, L. M., et al., Death of Military Working Dogs Due to Bartonella vinsonii Subspecies berkhoffii Genotype III Endocarditis and Myocarditis. Mil Med, 2017. 182(3): p. e1864-e1869.
39. Ghashghaei, R., et al., Bartonella endocarditis. J Cardiol Cases, 2016. 13(1): p. 1-3.
40. Ohad, D. G., et al., Molecular detection of Bartonella henselae and Bartonella koehlerae from aortic valves of Boxer dogs with infective endocarditis. Vet Microbiol, 2010. 141(1-2): p. 182-5.
41. Lin, E. Y., et al., Candidatus Bartonella mayotimonensis and endocarditis. Emerg Infect Dis, 2010. 16(3): p. 500-3.
42. Maillard, R., et al., Endocarditis in cattle caused by Bartonella bovis. Emerg Infect Dis, 2007. 13(9): p. 1383-5.
43. Weinborn-Astudillo, R. M., et al., Bartonella Infection in Stray Dogs from Central and Southern Chile (Linares and Puerto Montt). Vector Borne Zoonotic Dis, 2019.
44. Chondrogiannis, K., et al., Seronegative cat-scratch disease diagnosed by PCR detection of Bartonella henselae DNA in lymph node samples. Braz J Infect Dis, 2012. 16(1): p. 96-9.
45. Weinspach, S., et al., Cat scratch disease—heterogeneous in clinical presentation: five unusual cases of an infection caused by Bartonella henselae. Klin Padiatr, 2010. 222(2): p. 73-8.
46. Boillat, N. and G. Greub, [Cat scratch disease and other human infections caused by Bartonella species]. Rev Med Suisse, 2008. 4(152): p. 901-7.
47. Corral, J., et al., First Report of Bacillary Angiomatosis by Bartonella elizabethae in an HIV-Positive Patient. Am J Dermatopathol, 2019. 41(10): p. 750-753.
48. Orsag, J., et al., Cutaneous bacillary angiomatosis due to Bartonella quintana in a renal transplant recipient. Transpl Int, 2015. 28(5): p. 626-31.
49. Mitchell, B. M. and R. L. Font, Molecular detection of Bartonella henselae for the diagnosis of cat scratch disease and bacillary angiomatosis of the conjunctiva. Cornea, 2011. 30(7): p. 807-14.
50. Wong, R., J. Tappero, and C. J. Cockerell, Bacillary angiomatosis and other Bartonella species infections. Semin Cutan Med Surg, 1997. 16(3): p. 188-99.
51. Breitschwerdt, E. B., et al., Bartonella henselae Bloodstream Infection in a Boy With Pediatric Acute-Onset Neuropsychiatric Syndrome. J Cent New Syst Dis, 2019. 11: p. 1179573519832014.
52. Mascarelli, P. E., et al., Bartonella henselae infection in a family experiencing neurological and neurocognitive abnormalities after woodlouse hunter spider bites. Parasit Vectors, 2013. 6: p. 98.
53. Breitschwerdt, E. B., et al., Hallucinations, sensory neuropathy, and peripheral visual deficits in a young woman infected with Bartonella koehlerae. J Clin Microbiol, 2011. 49(9): p. 3415-7.
54. Breitschwerdt, E. B., et al., Bartonella vinsonii subsp. berkhoffii and Bartonella henselae bacteremia in a father and daughter with neurological disease. Parasit Vectors, 2010. 3(1): p. 29.
55. Andersson, S. G. and V. A. Kempf, Host cell modulation by human, animal and plant pathogens. Int J Med Microbiol, 2004. 293(7-8): p. 463-70.
56. Franz, B. and V. A. Kempf, Adhesion and host cell modulation: critical pathogenicity determinants of Bartonella henselae. Parasit Vectors, 2011. 4: p. 54.
57. Arvand, M., et al., Bartonella henselae-specific cell-mediated immune responses display a predominantly Th1 phenotype in experimentally infected C57BL/6 mice. Infect Immun, 2001. 69(10): p. 6427-33.
58. Il'ina, T. S. and V. N. Bashkirov, [Interaction of bacteria of the genus Bartonella with the host: inhibition of apoptosis, induction of proliferation, and formation of tumors]. Mol Gen Mikrobiol Virusol, 2008(3): p. 3-11.
59. Pulliainen, A. T. and C. Dehio, Bartonella henselae: subversion of vascular endothelial cell functions by translocated bacterial effector proteins. Int J Biochem Cell Biol, 2009. 41(3): p. 507-10.
60. Pulliainen, A. T. and C. Dehio, Persistence of Bartonella spp. stealth pathogens: from subclinical infections to vasoproliferative tumor formation. FEMS Microbiol Rev, 2012. 36(3): p. 563-99.
61. Resto-Ruiz, S., A. Burgess, and B. E. Anderson, The role of the host immune response in pathogenesis of Bartonella henselae. DNA Cell Biol, 2003. 22(6): p. 431-40.
62. Chomel, B. B., et al., Ecological, fitness and strategies of adaptation of Bartonella species to their hosts and vectors. Vet Res, 2009. 40(2): p. 29.
63. Maggi, R. G., A. W. Duncan, and E. B. Breitschwerdt, Novel chemically modified liquid medium that will support the growth of seven bartonella species. J Clin Microbiol, 2005. 43(6): p. 2651-5.
64. Hong, J., et al., Lymphatic Circulation Disseminates Bartonella Infection Into Bloodstream. J Infect Dis, 2017. 215(2): p. 303-311.
65. Wolf, A. L. N. A. C. R. G. M. E. B. B., In Pursuit of a Stealth Pathogen: Laboratory Diagnosis of Bartonellosis. Clinical Microbiology Newsletter, 2014. 36(5): p. 33-38.
66. Harms, A. and C. Dehio, Intruders below the radar: molecular pathogenesis of Bartonella spp. Clin Microbiol Rev, 2012. 25(1): p. 42-78.
67. Lynch, T., J. Iverson, and M. Kosoy, Combining culture techniques for Bartonella: the best of both worlds. J Clin Microbiol, 2011. 49(4): p. 1363-8.
68. Riess, T., et al., Analysis of a novel insect-cell culture medium-based growth medium for Bartonella species. Appl Environ Microbiol, 2008.
69. Weeden, A. L., et al., Bartonella henselae in canine cavitary effusions: prevalence, identification, and clinical associations. Vet Clin Pathol, 2017. 46(2): p. 326-330.
70. Mochizuki, H., S. G. Shapiro, and M. Breen, Detection of BRAF Mutation in Urine DNA as a Molecular Diagnostic for Canine Urothelial and Prostatic Carcinoma. PLoS One, 2015. 10(12): p. e0144170.
71. Mochizuki, H., et al., Genomic profiling of canine mast cell tumors identifies DNA copy number aberrations associated with KIT mutations and high histological grade. Chromosome Res, 2017. 25(2): p. 129-143.
72. Maggi, R. G., et al., Borrelia species in Ixodes affinis and Ixodes scapularis ticks collected from the coastal plain of North Carolina. Ticks Tick Borne Dis, 2010. 1(4): p. 168-71.

### TABLES

TABLE 1
Known DNA amplification inhibitory substances. Source: Promega
Corporation (Bessetti, J. An Introduction to PCR Inhibitors. Promega
Corporation. 2007. 2).

| Inhibitor | Source | Inhibitor | Source |
|---|---|---|---|
| Bile Salts | feces | Urea | urine |
| Polysaccharides | feces/plants | Hemoglobin | blood |
| Collagen | ti ssues | Lactoferrin | blood |
| Heme | blood | Immunoglobulin G | blood |
| Humic Acid | soil/plants | Calcium | milk/bones |
| Melanin | skin/hair | EDTA | blood tubes |
| Myoglobin | muscle | High DNA | tissues |
| Proteinases | milk | concentration | |
| | | Heparin | blood tubes |

TABLE 2
Bartonella species detected via ddPCR. Single species/strains: Bartonella DNA from
each of the below species/strains was singly spiked into naive human blood at either 0.10 pg/ul
(50 bacteria/ul) or 0.01 pg/ul (5 bacteria/ul): B. henselae Houston 1 (HI); B. henselae San
Antonio 2 strain (SA2); B. quintana Fuller; B.v. berkhoffii genotype 1; B.v. berkhoffii genotype
11; B.v. berkhoffii genotype ill; B.v. berkhoffii genotype IV*; B. vinsonii; B. melophagi; B.
volans; B. monaki; B. alsatica*; B. bovis; B. elizabethae; B. clarridgeiae; B. koehlerae. Dual
species: DNA of two Bartonella spp. spiked in naive human blood at equal (0.1 pg/ul)
concentrations. (*species DNA extracted from clinical cases).

| Single species-strains | | Dual species |
|---|---|---|
| B. henselae HI | B. clarridgeiae | B. henselae SA2 + B. quintana |
| B. henselae SA2 | B. melophagi | B. henselae SA2 + B. koehlerae |
| B. quintana Fuller | B. volans | B. henselae SA2 + B. v. berkhoffii TII |
| B. v. berkhoffii genotype I | B. elizabethae | B. quintana + B. v. berkhoffii TII |
| B. v. berkhoffii genotype II | B. monaki | B. quintana + B. koehlerae |
| B. v. berkhoffii genotype III | B. v. vinsonii | B. koehlerae + B. v. berkhoffii TII |
| B. v. berkhoffii genotype IV* | B. bovis | |
| B. koehlerae | B. alsatica* | |

Abbreviation: v. = vinsonii

TABLE 3
ddPCR primers and probes for Bartonella and housekeeping amplification.

| Primer/Probe | Name | Sequence |
|---|---|---|
| Bspp sense | BsppITS325s | 5′ CCTCAGATGATGATCCCAAGCCTTCTGGCG 3′ |
| | | (SEQ ID NO: 1) |
| Bspp antisense | BsppITS543as | 5′ TAAAYTGGTGGGCCTGGGAGGACTTG 3′ |
| | | (SEQ ID NO: 11) |
| Bspp probe | BsppITS500 | 5′ FAM-GTTAGAGCGCGCGCTTGATAAG-IABkFQ 3′ |
| | | (SEQ ID NO: 3) |
| HK sense | Hs-HMBS-90s | 5′ TTCCTTCCCTGAAGGGATTCACTCAG 3′ |
| | | (SEQ ID NO: 12) |
| HK antisense | Hs-HMBS-296as | 5′ TTAAGCCCAGCAGCCTATCTGACACCC 3′ |
| | | (SEQ ID NO: 13) |
| HK probe | Hs-HMBS150 | 5′ HEX-GAAAAGCCTGTTTACCAAGGAGCTTGAACATG- |
| | | BHQ-2 3′(SEQ ID NO: 14) |

TABLE 4
ddPCR signal reproducibility obtained with 0.05, 0.005, or 0.001 pg/ul of B. henselae
SA2 DNA spiked into naive human blood. The standard spiked samples were assayed once
weekly over a four-week interval (n = 4 runs/sample).

| Bacteria | | | | ddPCR | ddPCR |
|---|---|---|---|---|---|
| concentration | Bacteria per | Bacteria per | ITS copies | Average pos. | Standard |
| (pg/ul) | microliter | reaction | per reaction | drops | deviation |
| 0.05 | 25 | 125 | 250 | 213.0 | 29.0 |
| 0.005 | 2.5 | 12.5 | 25 | 22.0 | 4.0 |
| 0.001 | 0.5 | 2.5 | 5 | 4.0 | 1.0 |

TABLE 5
Intracellular ratio of bacteria per host cell during MCF10A epithelial
cell in vitro infection with B. henselae SA2 atMOIs of 1:1; 10:1; 30:1,
and 100:1 (intracellular bacteria/host cell ratio measured at 24 hrs,
48 hrs, and 72 hrs post infection).

| | | Bacteria/Cell |
|---|---|---|
| MOI | Time (hrs) | ddPCR |
| 1:1 | 24 | 0.14 |
| | 48 | 0.05 |
| | 72 | 0.02 |
| 10:1 | 24 | 0.6 |
| | 48 | 0.2 |
| | 72 | 0.1 |
| 30:1 | 24 | 4.8 |
| | 48 | 0.9 |
| | 72 | 0.8 |
| 100:1 | 24 | 0.9 |
| | 48 | 1.3 |
| | 72 | 1 |

TABLE 6
ddPCR primers and probes for Bartonella and Borrelia amplification.

| Primer/Probe | Name | Sequence |
|---|---|---|
| Borrelia sense | BobuITS120s | 5′ AGGTCATTTTGGGGGTTTAGCTCAGTTGGCT 3′ |
| | | (SEQ ID NO: 4) |
| Borrelia | BoLymeITS200as-m | 5′ AATGGAGGTTAAGGGACTCGAACCCT 5′ |
| antisense | | (SEQ ID NO: 5) |
| Borrelia probe | BobuITS160 | 5′ FAM-CGGCTTTGCAAGCCGAGGGTCAAG-IABkFQ 3′ |
| FAM | | (SEQ ID NO: 6) |
| Borrelia probe | BobuITS160 | 5′ HEX-CGGCTTTGCAAGCCGAGGGTCAAG-3BHQ-2 3′ |
| HEX | | (SEQ ID NO: 10) |
| Bartonella sense | BsppITS325s | 5′ CCTCAGATGATGATCCCAAGCCTTCTGGCG 3′ |
| | | (SEQ ID NO: 1) |
| Bartonella | BsppITS543as | 5′ TAAAYTGGTGGGCCTGGGAGGACTTG 3′ |
| antisense | | (SEQ ID NO: 2) |
| Bartonella | BsppITS500 | 5′ FAM-GTTAGAGCGCGCGCTTGATAAG-IABkFQ 3′ |
| probe | | (SEQ ID NO: 3) |

The foregoing is illustrative of the present inventive concept and is not to be construed as limiting thereof. Further embodiments of the present inventive concept are exemplified in the attached claims.

1-13. (canceled)

14. A method of identifying a fastidious microorganism in a sample comprising:
performing a nucleic acid amplification for nucleic acid sequences associated with a fastidious microorganism species or genus on a sample obtained from a subject suspected of being infected by a fastidious microorganism, wherein performing the nucleic acid amplification comprises distributing nucleic acids from the sample into a plurality of partitions, and performing nucleic acid amplification on the plurality of partitions, wherein the plurality of partitions is generated using a microfluidic process, a droplet generating process, or a droplet generating process using water-in-oil droplets (droplet digital PCR (ddPCR)); and
analyzing nucleic acid amplification products derived from the nucleic acid amplification,
wherein analysis of the nucleic acid amplification products provides information regarding identity of the fastidious microorganism infecting the subject.

15. The method of claim 14, wherein obtaining the sample comprises a nucleic acid extraction prior to performing the nucleic acid amplification.

16. (canceled)

17. The method of claim 14, wherein analysis of the nucleic acid amplification products to identify the fastidious microorganism comprises analyzing a partition distribution of the nucleic acid amplification products, and comparing the partition distribution of the nucleic acid amplification product with a known reference distribution for a particular fastidious microorganism.

18. The method of claim 14, wherein the nucleic acid amplification comprises amplification with primers directed to an internal transcribed spacer (ITS) between 16S and 23S rRNA genes of the fastidious microorganism.

19. The method of claim 14, wherein detecting whether the nucleic acid amplification product is produced comprises using an oligonucleotide probe for the nucleic acid amplification product, wherein the oligonucleotide probe is tagged with a fluorescent label.

20. The method of claim 14, wherein the fastidious microorganism is selected from the group consisting of Bartonella spp., Borrelia spp., Anaplasma spp., Ehrlichia spp., Babesia/Theileria spp., and Rickettsia spp.

21. (canceled)

22. The method of claim 20, wherein the fastidious microorganism is Bartonella spp., selected from the group consisting of B. henselae, B. quintana, B. alsatica, B. elizabethae, B. koehlerae, B. vinsonii subsp. berkhoffii genotype I, B. vinsonii subsp. berkhoffii genotype II, B. vinsonii subsp. berkhoffii genotype III, B. vinsonii subsp. berkhoffii genotype IV, B. vinsonii subsp. vinsonii, B. bovis, B. chomelii, B. melophagi, B. rochalimae, B. tribocorum, B. B. tamiae, B. Volans, and B. clarridgeiae.

23. (canceled)

24. The method of claim 20, wherein the fastidious microorganism is Borrelia spp., selected from the group consisting of B. burgdorferi, B. bissettii, B. garinii, B. mayonii, B. hermsii, B. parkeri, B. turicatae, B. miyamotoi, B. coriaceae, B. lusitanae, B. bavariensis, B. spielmanii, B. turcica, and B. afzelii.

25. The method of claim 14, wherein the subject is a mammalian subject.

26-27. (canceled)

28. The method of claim 14, wherein obtaining the sample from the subject comprises culturing an aliquot of body fluid or tissue drawn from the subject in a culture medium for growing fastidious microorganisms, prior to performing the nucleic acid amplification.

29. The method of claim 28, wherein the culture medium for growing fastidious microorganisms comprises an insect or mammalian cell culture media, and optionally comprises a supplement selected from the group consisting of β-Nicotinamide adenine dinucleotide (NAD), β-Nicotinamide adenine dinucleotide phosphate (NADPH), sodium pyruvate; adenosine 5′-triphosphate (ATP), essential amino acids, yeast extract, sodium bicarbonate, saponin, sodium polyanethol sulfonate (SPS), and fetal, horse, or human serum, or any combination thereof,
wherein the culture medium is protein-free.

30. The method of claim 28, wherein the aliquot of body fluid is selected from the group consisting of blood, plasma, serum, urine, cerebrospinal fluid, pleural fluid, pulmonary mucus, sputum, transudates, modified transudates, exudates, chest fluid, abdominal fluid, synovial fluid, peritoneal fluid, lymph, and effusions.

31. A method of detecting a fastidious microorganism in a sample derived from a subject comprising:
performing a nucleic acid amplification for a nucleic acid sequence that can identify the presence of the fastidious microorganism on nucleic acids extracted from the sample; and
detecting whether a nucleic acid amplification product is produced in the nucleic acid amplification,
wherein performing the nucleic acid amplification and detecting whether a nucleic acid amplification product is produced comprises analysis of a plurality of partitions generated using a microfluidic process, a droplet generating process, or a droplet generating process using water-in-oil droplets (droplet digital PCR (ddPCR)), and
wherein presence of the nucleic acid amplification product is indicative of the fastidious microorganism being present in the subject.

32. The method of claim 31, wherein the nucleic acid amplification comprises amplification with primers directed to an internal transcribed spacer (ITS) between 16S and 23S rRNA genes of the fastidious microorganism.

33. The method of claim 31, wherein detecting whether the nucleic acid amplification product is produced comprises using an oligonucleotide probe for the nucleic acid amplification product, wherein the oligonucleotide probe is tagged with a fluorescent label.

34. The method of claim 31, wherein the fastidious microorganism is selected from the group consisting of Bartonella spp., Borrelia spp., Anaplasma spp., Ehrlichia spp., Babesia/Theileria spp., and Rickettsia spp.

35. (canceled)

36. The method of claim 33, wherein the fastidious microorganism is Bartonella spp., selected from the group consisting of B. henselae, B. quintana, B. alsatica, B. elizabethae, B. koehlerae, B. vinsonii subsp. berkhoffii genotype I, B. vinsonii subsp. berkhoffii genotype II, B. vinsonii subsp. berkhoffii genotype III, B. vinsonii subsp. berkhoffii genotype IV, B. vinsonii subsp. vinsonii, and B. clarridgeiae.

37-55. (canceled)

56. The method of claim 31, wherein nucleic acids that detect and/or identify a presence of the fastidious microorganism are captured and/or concentrated prior to performing the nucleic acid amplification.

57. The method of claim 56, wherein capturing and/or concentrating of the nucleic acid sequence or nucleic acid sequences comprises using highly porous hydrogel particles having high affinity for nucleic acids associated with the fastidious microorganism.

58. (canceled)

59. The method of claim 57, wherein the highly porous hydrogel particles comprise NANOTRAP® particles and/or NANOTRAP® magnetic particles.
